(19) United States
(12) Patent Application Publication
(10) Pub. No.: US 2007/0270361 A1
(43) Pub. Date: Nov. 22, 2007

(54) Sars Nucleic Acids, Proteins, Vaccines, and Uses Thereof

(76) Inventors: Shan Lu, Franklin, MA (US); Te-Hui W. Chou, Wayland, MA (US); Shixia Wang, Northborough, MA (US)

Correspondence Address: FISH & RICHARDSON PC, P.O. BOX 1022, MINNEAPOLIS, MN, 55440-1022, US

(21) Appl. No.: 10/565,314

(22) PCT Filed: Aug. 4, 2004
(86) PCT No.: PCT/US04/25372

§ 371 Date: Apr. 11, 2007

Related U.S. Application Data
(60) Provisional application No. 60/492,523, filed on Aug. 4, 2003.

(51) Int. Cl. A61K 31/70; A61K 38/00; A61P 11/00; C07H 21/04; C07K 16/00; C12N 5/00; C12N 5/08; C12P 21/06

(52) U.S. Cl. 514/44; 435/325; 435/366; 435/69.1; 530/300; 530/391.1; 536/23.5

(57) ABSTRACT

Codon-optimized nucleic acids, proteins, vaccines, and antibodies are provided herein.

### CROSS-REFERENCE TO RELATED APPLICATIONS

This application claims the benefit of priority of U.S. Ser. No. 60/492,523, filed Aug. 4, 2003, the contents of which are hereby incorporated by reference in its entirety.

The work described herein was funded by Grants AI 40337 and AI 44338 from the National Institutes of Health, Institute of Allergy and Infectious Diseases. The United States government may, therefore, have certain rights in the invention.

### TECHNICAL FIELD

This invention relates to viral nucleic acids sequences, proteins, and subunit (both nucleic acid and recombinant protein) vaccines and more particularly to viral nucleic acids sequences that have been optimized for expression in mammalian host cells.

### BACKGROUND

Severe Acute Respiratory Syndrome (SARS) is an emerging infectious illness with a tendency for rapid spread from person to person (MMWR Morb Mortal Wkly Rep, 52 (12): 255-6, 2003; MMWR Morb Mortal Wkdy Rep, 52 (12): 241-6, 248, 2003; Lee N et al., N Engl J Med, 348(20): 1986-94, 2003; Poutanen et al., N Engl J Med, 348(20): 1995-2005, 2003). A newly identified coronavirus is now established as the etiologic agent (Drosten et al., N Engl J Med, 348(20): 1967-76, 2003; Ksiazek et al., N Engl J Med, 348(20): 1953-66, 2003). Coronaviruses have characteristic surface peplomer spikes formed by oligomers of the surface S-glycoprotein. The S-proteins are the principal targets for neutralizing antibodies (Saif, Vet Microbiol, 37(34): 285-97, 1993). The protective efficacy of humoral immunity has been demonstrated in several animal models of coronavirus disease (e.g., avian infectious bronchitis virus disease and respiratory bovine coronavirus disease) (Lin et al., Clin Diagn Lab Immunol 8 (2): 357-62, 2001; Mondal and Naqi, Vet Immunol Inmunopathol, 79 (1-2): 31-40, 2001; Wang et al., Avian Dis, 46 (4): 831-8, 2002.18).

The recently published sequence of the human SARS corona virus (human SARS-CoV) reveals that it represents a new strain (Drosten et al., N Engl J Med, 348(20): 1967-76, 2003; Ksiazek et al., N Engl J Med, 348(20): 1953-66, 2003). While it is seroreactive with some antisera and monoclonal antibodies to group 1 coronaviruses, it appears to be best classified as a fourth serogroup given its sequence divergence from other strains. Neutralization with available antibodies has not been reported. With the rapid spread of the SARS epidemic and a mortality rate of 5% and higher for aged individuals, it is crucial to develop therapeutic and prophylactic agents. The most severe clinical outcomes of this infection have been associated with prolonged viremia (Drosten et al., N Engl J Med, 348(20): 1967-76, 2003).

Laboratory analyses of convalescent serum samples from individuals with probable SARS have shown high levels of specific reactivity with infected cells and conversion from negative to positive reactivity or diagnostic rises in the indirect fluorescence antibody test (Ksiazek et al., N Engl J Med, 348(20): 1953-66, 2003). In contrast, sera from United States blood donors and persons with known HCV 229E or OC43 infection were negative for antibodies to this novel coronavirus. These results indicate that this virus has not been widely circulated in human populations (Ksiazek et al., N Engl J Med, 348(20): 1953-66, 2003).

### SUMMARY

The present invention is based, in part, on the observation that codon-optimized variant forms of nucleic acids encoding the SARS-CoV spike glycoprotein (S protein), membrane protein (M protein), envelope protein (E protein), and nucleocapsid protein (N protein) can be used to express the proteins in appropriate host cells. Enhanced expression can provide large quantities of SARS proteins and fragments thereof for diagnostic and therapeutic applications. Nucleic acids encoding SARS-CoV antigens that are efficiently expressed in mammalian host cells are useful, e.g., for inducing immune responses to the antigens in the host. Production of viral proteins in mammalian cells can provide SARS proteins that fold properly, oligomerize with natural binding partners, and/or possess native post-translational modifications such as glycosylation. These features can enhance immunogenicity, thereby increasing protection afforded by vaccination with the proteins (or with the nucleic acids encoding the proteins). Codon-optimized nucleic acids can be constructed by synthetic means, obviating the need to obtain nucleic acids from live virus, thus decreasing the risks associated with working with SARS-CoV.

In one aspect, the invention features an isolated nucleic acid including: a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, wherein the sequence has been codon-optimized for expression in a mammalian host (e.g., a human host, e.g., wherein the sequence is synthetic or artificial).

In one embodiment, the sequence encodes a SARS Co-V S polypeptide or fragment thereof, wherein the sequence (or fragment thereof) comprises at least 80%, 85%, 90%, 95%, 96%, 97%, 98%, 99%, or 100% identity with the sequence set forth in SEQ ID NO:1 (or corresponding fragment of SEQ ID NO:1, e.g., a fragment encoding amino acids 1-535 or 11-535 of the S protein). In one embodiment, the sequence encodes a leader peptide that is or is not naturally associated with the S polypeptide (e.g., a heterologous leader peptide). In one embodiment, the sequence encodes a tPA leader peptide (or another leader peptide which can improve the expression or secretion of the polypeptide).

In one embodiment, the sequence encodes an extracellular portion of the S polypeptide (e.g., amino acids 1-1190 of SEQ ID NO:2, or a portion lacking the putative leader peptide, e.g., amino acids 12-1190 of SEQ ID NO:2).

In another aspect, the invention features an isolated nucleic acid including: a sequence encoding a SARS-CoV M polypeptide, or fragment thereof, wherein the sequence comprises at least 80%, 85%, 90%, 95%, 96%, 97%, 98%, 99%, or 100% with the sequence set forth in SEQ ID NO:19.

In another aspect, the invention features an isolated nucleic acid including: a sequence encoding a SARS-CoV E polypeptide, or fragment thereof, wherein the sequence comprises at least 80%, 85%, 90%, 95%, 96%, 97%, 98%, 99%, or 100% identity with the sequence set forth in SEQ ID NO:21.

In another aspect, the invention features an isolated nucleic acid including: a sequence encoding a SARS-CoV N polypeptide, or fragment thereof, wherein the sequence comprises at least 80%, 85%, 90%, 95%, 96%, 97%, 98%, 99%, or 100% identity with the sequence set forth in SEQ ID NO:23.

In another aspect, the invention features a nucleic acid expression vector including: a sequence encoding a SARS-CoV S polypeptide, M polypeptide, E polypeptide, N polypeptide, or fragment thereof, wherein the sequence is codon-optimized for expression in a host cell.

In another aspect, the invention features a composition including an isolated nucleic acid, wherein the isolated nucleic acid comprises (a) a codon-optimized sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof; (b) a start codon immediately upstream of the nucleotide sequence; (c) a mammalian promoter operably linked to the codon-optimized sequence; and (d) a mammalian polyadenylation signal operably linked to the nucleotide sequence, wherein the promoter directs transcription of mRNA encoding the SARS-CoV polypeptide. The composition can further include an adjuvant. In one embodiment, the mammalian promoter is a cytomegalovirus immediate-early promoter.

In one embodiment, the polyadenylation signal is derived from a bovine growth hormone gene. In one embodiment, the composition further includes a pharmaceutically acceptable carrier. In one embodiment, the composition further includes particles to which the isolated nucleic acid is bound, wherein the particles are suitable for intradermal, intramuscular or mucosal administration.

In another aspect, the invention features an isolated cell including a nucleic acid described herein.

In another aspect, the invention features an isolated polypeptide encoded by a nucleic acid described herein.

In another aspect, the invention features an isolated antibody or antigen binding fragment thereof that specifically binds to a polypeptide described herein, e.g., a SARS protein.

In another aspect, the invention features a method for making a SARS-CoV polypeptide, the method including: constructing a nucleic acid, wherein the nucleic acid comprises a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, and wherein the codons encoding the polypeptide are optimized for expression in a host cell, expressing the nucleic acid in the host cell under conditions that allow the polypeptide to be produced, and isolating the polypeptide.

In another aspect, the invention features a method for inducing an immune response to SARS-CoV polypeptide in a subject, the method including: administering to the subject a composition including an isolated nucleic acid, wherein the isolated nucleic acid comprises (a) a codon-optimized sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof; (b) a start codon immediately upstream of the nucleotide sequence; (c) mammalian promoter operably linked to the codon-optimized sequence; and (d) a mammalian polyadenylation signal operably linked to the nucleotide sequence, wherein the promoter directs transcription of mRNA encoding the SARS-CoV polypeptide, wherein the composition is administered in an amount sufficient for the nucleic acid to express the SARS-CoV polypeptide at a level sufficient to induce an immune response against SARS in the subject.

The invention also features nucleic acids comprising a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, for inducing an immune response to the SARS-CoV polypeptide in a subject, wherein the sequence has been codon-optimized for expression in the subject. The nucleic acid can include a codon-optimized nucleic acid sequence described herein (e.g., a codon-optimized DNA sequence encoding the S protein or a fragment thereof, e.g., comprising all or a portion of SEQ ID NO:1).

The invention also features the use of a nucleic acid comprising a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, for the manufacture of a medicament for inducing an immune response to the SARS-CoV polypeptide in a subject, wherein the sequence has been codon-optimized for expression in the subject. The nucleic acid can include a codon optimized nucleic acid sequence described herein (e.g., a codon-optimized DNA sequence encoding the S protein or a fragment thereof, e.g., comprising all or a portion of SEQ ID NO:1).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a representation of the SARS-CoV Spike glycoprotein and codon-optimized S proteins encoded by nucleic acid constructs described herein. “tPA” refers to the tissue plasminogen leader sequence. “TM” refers to a transmembrane domain. “dTM” indicates that a protein lacks a transmembrane domain. S1, S2, S1.1, S1.2 are fragments of the S protein. “ACE2 R” refers to the angiotensin-converting enzyme 2 receptor binding domain on the S protein.

FIG. 2 is a graph depicting the results of assays to determine binding of antisera from rabbits immunized with a codon-optimized DNA vectors encoding the wt-S protein, tPA-S.dTM, or vector alone. Arrows indicate the time points at which animals were administered DNA.

FIGS. 3A and 3B are a set of graphs depicting the results of assays to determine reactivity of antisera from rabbits immunized with codon-optimized DNA vectors encoding tPA-S.dTM, tPA-S1.1, tPA-S1.2, tPA-S2.dTM, or vector. In FIG. 3A, reactivity to tPA-S protein was measured. In FIG. 3B, reactivity to tPA-S1.2 was measured.

FIG. 4A is a representation of SDS-PAGE and Western blot analysis of S protein antigens expressed by various codon-optimized DNA constructs probed with antisera from rabbits immunized with codon-optimized DNA encoding tPA-S.dTM.

FIG. 4B is a representation of SDS-PAGE and Western blot analysis of S protein antigens expressed by various codon-optimized DNA constructs probed with antisera from rabbits immunized with codon-optimized DNA encoding tPA-S1.1.

FIG. 4C is a representation of SDS-PAGE and Western blot analysis of S protein antigens expressed by various codon-optimized DNA constructs probed with antisera from rabbits immunized with codon-optimized DNA encoding tPA-S1.2.

FIG. 4D is a representation of SDS-PAGE and Western blot analysis of S protein antigens expressed by various codon-optimized DNA constructs probed with antisera from rabbits immunized with codon-optimized DNA encoding tPA-S2.dTM.

FIG. 4E is a representation of SDS-PAGE and Western blot analysis of S protein antigens expressed by various codon-optimized DNA constructs probed with antisera against the S protein. A subset of S protein antigens analyzed were treated with urea prior to SDS-PAGE.

FIG. 5 is a representation of SDS-PAGE and Western blot analysis of lysed SARS-CoV stocks or uninfected Vero E6 cells, probed with antisera raised in rabbits immunized with codon-optimized DNA encoding various S protein fragments. LMP: low molecular weight products, and HMC: high molecular weight complex. S: expected fully glycosylated Spike protein.

FIGS. 6A-6C are a set of pictures of culture plates containing mock-infected Vero E6 cells (FIG. 6A), SARS-CoV infected Vero E6 cells, 4 days after infection (FIG. 6B), and SARS-CoV infected Vero E6 cells cultured in the presence of antisera raised in rabbits immunized with codon-optimized DNA encoding the S protein.

FIG. 7 is a graph depicting the results of assays to determine the neutralizing antibody titer in antisera raised in rabbits immunized with various codon-optimized DNA constructs encoding S protein fragments (or vector alone).

FIGS. 8A-8B are a set of graphs depicting percent neutralization of SARS-CoV by antisera raised in rabbits immunized with various codon-optimized DNA constructs encoding S protein fragments. FIG. 8A depicts results of assays in which antisera from animals immunized with tPA-S.dTM, TPA-S1, tPA-S2.dTM, or vector alone was tested. FIG. 8B depicts results of assays in which antisera from animals immunized with TPA-S1.1, TPA-S1.2, or pre-bleed sera was tested.

FIG. 9 is a representation of SDS-PAGE and Western blot analysis of various fragments of S protein and S protein associated with SARS-CoV virions were examined. A subset of protein samples were treated with N-glycosidase F (PNGase F) prior to SDS-PAGE.

FIGS. 10A and 10B are a representation of a codon-optimized nucleotide sequence encoding the full-length SARS-CoV S protein.

FIG. 11 is a representation of the amino acid sequence of the full-length SARS-Co V S protein.

FIG. 12 is a representation of a codon optimized nucleotide sequence encoding amino acids 1-535 of the SARS-CoV S protein.

FIG. 13 is a representation of a codon-optimized nucleotide sequence encoding amino acids 1-535 of the SARS-CoV S protein. Nucleotides (NT) 1-96 encode the tPA leader sequence; NT 97-1608 encode a portion of the S protein.

FIG. 14 is a representation of a codon-optimized nucleotide sequence encoding amino acids 534-798 of the SARS-CoV S protein. NT 1-96 encode the tPA leader sequence; NT 97-804 encode a portion of the S protein.

FIG. 15 is a representation of a codon-optimized nucleotide sequence encoding amino acids 797-1255 of the SARS-CoV S protein. NT 1-96 encode the tPA leader sequence; NT 97-1380 encode a portion of the S protein.

FIG. 16 is a representation of a codon-optimized nucleotide sequence encoding amino acids 1-222 of the SARS-CoV M protein.

FIG. 17 is a representation of a codon-optimized nucleotide sequence encoding amino acids 1-77 of the SARS-CoV E protein.

FIG. 18 is a representation of a codon-optimized nucleotide sequence encoding amino acids 1-424 of the SARS-CoV N protein.

FIGS. 19A-19B are a representation of the native nucleotide sequence of the SARS-CoV S protein (see also GenBank® Acc. No. AY278741).

FIG. 20 is a representation of the native nucleotide sequence of the SARS-CoV M protein (see also GenBank® Acc. No. AY278741).

FIG. 21 is a representation of the native nucleotide sequence of the SARS-CoV E protein (see also GenBank® Acc. No. AY278741).

FIG. 22 is a representation of the native nucleotide sequence of the SARS-CoV E protein (see also GenBank® Acc. No. AY278741).

FIG. 23 is a representation of the amino acid sequence encoded by SEQ ID NO:3.

FIG. 24 is a representation of the amino acid sequence encoded by SEQ ID NO:5.

FIG. 25 is a representation of the amino acid sequence encoded by SEQ ID NO:7.

FIG. 26 is a representation of the amino acid sequence encoded by SEQ ID NO:9.

FIG. 27 is a representation of the amino acid sequence encoded by SEQ ID NO:11.

FIG. 28 is a representation of the amino acid sequence encoded by SEQ ID NO:13,

FIG. 29 is a representation of the amino acid sequence encoded by SEQ ID NO:15.

FIG. 30 is a representation of the native SARS-CoV S protein amino acid sequence.

FIG. 31 is a representation of the native SARS-CoV M protein amino acid sequence.

FIG. 32 is a representation of the native SARS-CoV E protein amino acid sequence.

FIG. 33 is a representation of the native SARS-CoV N protein amino acid sequence.

Like reference symbols in the various drawings indicate like elements.

### DETAILED DESCRIPTION

Coronaviruses display peplomer spikes formed by oligomers of the surface S-glycoprotein. These proteins can mediate interaction of the viruses with receptors on host cells to allow entry and fusion, and also are major targets for neutralizing antibodies. Efficient expression of S proteins is useful for the preparation of therapeutic and diagnostic proteins and antibodies for, e.g., diagnosing, treating, preventing, and analyzing SARS coronaviruses. Other viral proteins are also useful for therapeutic and diagnostic purposes. For example, the membrane (M), envelope (E), and nucleocapsid (N) proteins can also be used in the study and treatment of coronaviruses. Each of these SARS viral antigens can functions as a component in a single-agent or multi-agent formulations of subunit-based SARS prophylactic vaccines

Provided herein are codon-optimized nucleic acid sequences that encode the SARS-CoV S, M, B, and N proteins and methods for the construction of such sequences. The invention also features nucleic acid vaccines that can express these proteins in a subject in sufficiently high concentrations to provide protective immunity against subsequent exposure to SARS. The expressed proteins themselves, methods of expressing the proteins can be used as recombinant protein SARS vaccines. These nucleic acid sequences and proteins can be used to generate antibodies that recognize the SARS proteins and fragments of the SARS proteins and the antibodies can be used in the diagnosis, prevention, and treatment of SARS.

In order that the present invention may be more readily understood, certain terms are first defined. Additional definitions are set forth throughout the detailed description.

A “subunit” vaccine is a vaccine whose active ingredient antigen is only part of a pathogen, e.g. one protein or a fragment of such protein in a pathogen with multiple proteins.

A “nucleic acid vaccine” is a vaccine whose active ingredient is at least one isolated nucleic acid that encodes a polypeptide antigen.

A “recombinant protein vaccine” is a vaccine whose active ingredient is at least one protein antigen that is produced by recombinant expression.

An “isolated nucleic acid” is a nucleic acid free of the genes that flank the gene of interest in the genome of the organism or virus in which the gene of interest naturally occurs. The term therefore includes a recombinant DNA incorporated into an autonomously expressing plasmid in mammalian systems. It also includes a separate molecule such as a cDNA, a genomic fragment, a fragment produced by polymerase chain reaction, or a restriction fragment. It also includes a recombinant nucleotide sequence that is part of a hybrid gene, i.e., a gene encoding a fusion protein. An isolated nucleic acid is substantially free of other cellular or viral material (e.g., free from the protein components of a viral vector), or culture medium when produced by recombinant techniques, or substantially free of chemical precursors or other chemicals when chemically synthesized.

Expression control sequences are “operably linked” when they are incorporated into other nucleic acid so that they effectively control expression of a gene of interest.

An “adjuvant” is a compound or mixture of compounds that enhances the ability of a nucleic acid vaccine to elicit an immune response.

A “mammalian promoter” is any nucleic acid sequence, regardless of origin, that is capable of driving transcription of a mRNA coding for a SARS protein within a mammalian cell.

A “mammalian polyadenylation signal” is any nucleic acid sequence, regardless of origin, that is capable of terminating transcription of an mRNA encoding a SARS protein within a mammalian cell.

The term “S protein” refers to the spike glycoprotein encoded by SARS-CoV. “Protein” is used interchangeably with “polypeptide”, and includes both proteins produced in vitro and proteins expressed in vivo after nucleic acid sequences are administered into the host animals or human subjects.” The predicted leader peptide corresponds to amino acids 1-11 of SEQ ID NO:18. The predicted ligand binding domain corresponds to amino acids 318-510 of SEQ ID NO:10. The predicted extracellular portion of the mature S protein corresponds to amino acids 12-1190 of SEQ ID NO:18, and is soluble and secreted by cells. The predicted transmembrane domain corresponds to amino acids 1192-1226 of SEQ ID NO:18. The predicted cytoplasmic domain corresponds to amino acids 1227-1255 of SEQ ID NO:18.

An “anti-SARS protein antibody” or “anti-SARS antibody” is an antibody that interacts with (e.g., binds to) a SARS protein. As used herein, the term “treat” or “treatment” is defined as the application as administration of a nucleic acid encoding a SARS-CoV S, M, E, or N protein, or fragment thereof, or anti-SARS antibodies to a subject, e.g., a patient, or application or administration to an isolated tissue or cell from a subject, e.g., a patient, which is returned to the patient. Proteins encoded by the nucleic acids, or antibodies that specifically bind to the proteins can also be administered. The nucleic acid can be administered alone or in combination with a second agent. The subject can be a patient having a disorder (e.g., a viral disorder, e.g., SARS), a symptom of a disorder, or a predisposition toward a disorder. The treatment can be to cure, heal, alleviate, relieve, alter, remedy, ameliorate, palliate, improve, or affect the disorder, or symptoms of the disorder.

As used herein, an amount of a nucleic acid, protein or an anti-SARS protein antibody effective to treat a disorder, or a “therapeutically effective amount,” refers to an amount that is effective, upon single or multiple dose administration to a subject, in treating a subject with an infection by SARS-CoV. As used herein, an amount of a nucleic acid, protein, or an anti-SARS protein antibody effective to prevent a disorder, or a “a prophylactically effective amount,” of the antibody refers to an amount which is effective, upon single- or multiple-dose administration to the subject, in preventing or delaying the occurrence of the onset or recurrence of a SARS disorder, or treating a symptom thereof.

As used herein, “specific binding” or “specifically binds to” refer to the ability of an antibody to: (1) bind to a SARS protein as shown by a specific biochemical analysis, such as a specific band in a Western Blot analysis, or (2) bind to a SARS protein with a reactivity that is at least two-fold greater than its reactivity for binding to an antigen (e.g., BSA, casein) other than a SARS protein.

As used herein, the term “antibody” refers to a protein including at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol., 196:901-917, which are incorporated herein by reference). Preferably, each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region includes three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., a mouse, framework modified to decrease antigenicity in humans, or a synthetic framework, e.g., a consensus sequence.

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to a portion of an antibody that specifically binds to a SARS protein (e.g., an S protein), e.g., a molecule in which one or more immunoglobulin chains is not full length, but which specifically binds to a SARS protein. Examples of binding fragments encompassed within the term “antigen-binding fragment” of an antibody include: (i) a Fab fragment, a monovalent fragment consisting of the VL, VH, CL, and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind to, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science, 242:423-426; and Huston et al. (1988) Proc. Natl. Acad. Sci. USA, 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The term “polyclonal antibody” refers to an antibody preparation, either as animal or human sera or as prepared by in vitro production, which can bind to more than one epitope on one SARS antigen or multiple epitopes on more than one antigen.

The term “recombinant” antibody, as used herein, refers to antibodies that are prepared, expressed, created, or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, in vitro generated (e.g., by phage display) antibodies, and may optionally include constant regions derived from human germline immunoglobulin sequences.

As used herein, the term “substantially identical” (or “substantially homologous”) refers to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the first antibody.

Calculations of “homology” or “identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In different embodiments, the length of a reference sequence aligned for comparison purposes is at least 50%, e.g., at least 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences are accomplished using a mathematical algorithm. The percent homology between two amino acid sequences is determined using the Needleman and Wunsch (1970), J. Mol. Biol., 48:444-453, algorithm which has been incorporated into the GAP program in the GCG software package, using a Blossum 62 scoring matrix with a gap penalty of 12, a gap extend penalty of 4, and a frameshift gap penalty of 5.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated herein by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5M sodium phosphate, 7% SDS at 65° C., followed by one or more washes at 0.2×SSC, 1% SDS at 65° C.

It is understood that the antibodies and antigen binding fragments thereof described herein may have additional conservative or non-essential amino acid substitutions, which do not have a substantial effect on the polypeptide functions. Whether or not a particular substitution will be tolerated, i.e., will not adversely affect desired biological properties, such as binding activity, can be determined as described in Bowie et al., (1990) Science, 247:1306-1310. A “conservative amino acid substitution” is one in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of a polypeptide, such as a binding agent, e.g., an antibody, without substantially altering a biological activity, whereas an “essential” amino acid residue results in such a change.

Construction of Optimized Sequences

Viral proteins and proteins that are naturally expressed at low levels can provide challenges for efficient expression by recombinant means. Viral proteins often display a codon usage that is inefficiently translated in a mammalian host cell. Alteration of the codons native to the viral sequence can facilitate more robust expression of these proteins. Codon preferences for abundantly-expressed proteins have been determined in a number of species, and can provide guidelines for codon substitution. HIV envelope and gag genes have been codon optimized to improve the expression of these viral antigens. Substitution of viral codons can be done by routine methods, such as site-directed mutagenesis, or construction of oligonucleotides corresponding to the optimized sequence by chemical synthesis. See, e.g., Mirzabekov et al., J Biol Chem., 274(40):28745-50, 1999.

The optimization should also include consideration of other factors that can affect synthesis of oligos and/or expression. For example, sequences that result in RNAs predicted to have a high degree of secondary structure are avoided. AT- and GC-rich sequences interfere with DNA synthesis and are also avoided. Other motifs that can be detrimental to expression include internal TATA boxes, chi-sites, ribosomal entry sites, procarya inhibitory motifs, cryptic splice donor and acceptor sites, and branch points. These sequences can be identified by computer software and they can be excluded when the codon optimized sequences are constructed manually.

Nucleic Acids, Vectors, and Host Cells

One aspect of the invention pertains to isolated nucleic acid, vector, and host cell compositions that can be used for recombinant expression of the optimized nucleic acid sequences and for vaccines.

In another aspect, the invention features host cells and vectors (e.g., recombinant expression vectors) containing the nucleic acids, e.g., the optimized sequences encoding SARS proteins, or a sequence encoding an anti-SARS protein antibody, or an antigen binding fragment thereof.

Prokaryotic or eukaryotic host cells may be used. The terms “host cell” and “recombinant host cell” are used interchangeably herein. Such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E. coli, or eukaryotic, e.g., insect cells, yeast, or mammalian cells (e.g., cultured cell or a cell line, e.g., a primate cell such as a Vero cell, or a human cell). Other suitable host cells are known to those skilled in the art.

In another aspect, the invention features a vector, e.g., a recombinant expression vector. The recombinant expression vectors of the invention can be designed for expression of the SARS proteins, anti-SARS protein antibodies, or an antigen-binding fragments thereof, in prokaryotic or eukaryotic cells. For example, new polypeptides described herein can be expressed in E. coli, insect cells (e.g., using baculovirus expression vectors), yeast cells, or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is often carried out in E. coli with vectors containing constitutive or inducible promoters directing the expression of either fusion or non-fusion proteins. Fusion vectors add a number of amino acids to protein or antibody encoded therein, usually to the constant region of a recombinant antibody.

A codon-optimized nucleic acid can be expressed in mammalian cells using a mammalian expression vector. Examples of mammalian expression vectors include pCDM8 (Seed, B. Nature 329:840, 1987) and pMT2PC Kaufman et al. EMBO J. 6:187-195, 1987). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd ed., Cold Spring Harbor Laboratory, Cold Spring Harbor Laboratory Press, Cold Spring Harbor, N.Y., 1989.

In one embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev., 1:268-277, 1987), lymphoid-specific promoters (Calame and Eaton, Adv. Immunol., 43:235-275, 1988), in particular promoters of T cell receptors (Winoto and Baltimore, EMBO J., 8:729-733, 1989) and immunoglobulins (Banerji et al., Cell, 33:729-740, 1983; Queen and Baltimore, Cell, 33:741-748, 1983), neuron-specific promoters (e.g., the neurofilament promoter; Byrne and Ruddle, Proc. Natl. Acad. Sci., USA 86:5473-5477, 1989), pancreas-specific promoters (Edlund et al., Science, 230:912-916, 1985), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, Science, 249:374-379, 1990 and the α-fetoprotein promoter (Campes and Tilghman, Genes Dev., 3:537-546, 1989).

In addition to the coding sequences, the new recombinant expression vectors described herein carry regulatory sequences that are operatively linked and control the expression of the proteins/antibody genes in a host cell.

Nucleic Acid Vaccines

A SARS polypeptide encoded by a codon-optimized nucleic acid used in the new methods or compositions is any protein or polypeptide sharing an epitope with a naturally occurring SARS protein, e.g., a SARS S, M, E, or N protein. The SARS polypeptides can differ from the wild type sequence by additions or substitutions within the amino acid sequence, and may preserve a biological function of the SARS polypeptide (e.g., receptor binding by the S protein). Amino acid substitutions may be made on the basis of similarity in polarity, charge, solubility, hydrophobicity, hydrophilicity, and/or the amphipathic nature of the residues involved.

Nonpolar (hydrophobic) amino acids include alanine, leucine, isoleucine, valine, proline, phenylalanine, tryptophan, and methionine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid.

Alteration of residues are preferably conservative alterations, e.g., a basic amino acid is replaced by a different basic amino acid.

The nucleic acids useful for inducing an immune response include at least three components: (1) a SARS protein coding sequence beginning with a start codon, (2) a mammalian transcriptional promoter operatively linked to the coding sequence for expression of the SARS protein, and (3) a mammalian polyadenylation signal operably linked to the coding sequence to terminate transcription driven by the promoter. In this context, a “mammalian” promoter or polyadenylation signal is not necessarily a nucleic acid sequence derived from a mammal. For example, it is known that mammalian promoters and polyadenylation signals can be derived from viruses.

The nucleic acid vector can optionally include additional sequences such as enhancer elements, splicing signals, termination and polyadenylation signals, viral replicons, and bacterial plasmid sequences. Such vectors can be produced by methods known in the art. For example, a nucleic acid encoding the desired SARS protein can be inserted into various commercially available expression vectors. See, e.g., Invitrogen Catalog, 1998. In addition, vectors specifically constructed for nucleic acid vaccines are described in Yasutomi et al., J Virol, 70:678-681 (1996).

Administration of Nucleic Acids

The new nucleic acids of the described herein can be administered to an individual, or inoculated, in the presence of substances that have the capability of promoting nucleic acid uptake or recruiting immune system cells to the site of the inoculation. For example, nucleic acids encapsulated in microparticles have been shown to promote expression of rotaviral proteins from nucleic acid vectors in vivo (U.S. Pat. No. 5,620,896).

A mammal can be inoculated with nucleic acid through any parenteral route, e.g., intravenous, intraperitoneal, intradermal, subcutaneous, intrapulmonary, or intramuscular routes. The new nucleic acid vaccines can also be administered, orally, by particle bombardment using a gene gun, or by other needle-free delivery systems. Muscle is a useful tissue for the delivery and expression of SARS protein-encoding nucleic acids, because mammals have a proportionately large muscle mass which is conveniently accessed by direct injection through the skin. A comparatively large dose of nucleic acid can be deposited into muscle by multiple and/or repetitive injections. Multiple injections can be used for therapy over extended periods of time.

Administration of nucleic acids by conventional particle bombardment can be used to deliver nucleic acid for expression of a SARS protein in skin or on a mucosal surface. Particle bombardment can be carried out using commercial devices. For example, the Accell II® (PowderJect® Vaccines, Inc., Middleton, Wis.) particle bombardment device, one of several commercially available “gene guns,” can be employed to deliver nucleic acid-coated gold beads. A Helios Gene Gun® (Bio-Rad) can also be used to administer the DNA particles. Information on particle bombardment devices and methods can be found in sources including the following: Yang et al., Proc Natl Acad Sci USA, 87:9568 (1990); Yang, CRC Crit Rev Biotechnol, 12:335 (1992); Richmond et al., Virology, 230:265-274 (1997); Mustafa et al., Virology, 229:269-278 (1997); Livingston et al., Infect Immun, 66:322-329 (1998) and Cheng et al., Proc Natl Acad Sci USA, 90:4455 (1993).

In some embodiments, an individual is inoculated by a mucosal route. The SARS protein-encoding nucleic acid can be administered to a mucosal surface by a variety of methods including nucleic acid-containing nose-drops, inhalants, suppositories, or microspheres. Alternatively, a nucleic acid vector containing the codon-optimized gene can be encapsulated in poly(lactide-co-glycolide) (PLG) microparticles by a solvent extraction technique, such as the ones described in Jones et al., Infect Immun, 64:489 (1996); and Jones et al., Vaccine, 15:814 (1997). For example, the nucleic acid is emulsified with PLG dissolved in dichloromethane, and this water-in-oil emulsion is emulsified with aqueous polyvinyl alcohol (an emulsion stabilizer) to form a (water-in-oil)-in-water double emulsion. This double emulsion is added to a large quantity of water to dissipate the dichloromethane, which results in the microdroplets hardening to form microparticles. These microdroplets or microparticles are harvested by centrifugation, washed several times to remove the polyvinyl alcohol and residual solvent, and finally lyophilized. The microparticles containing nucleic acid have a mean diameter of 0.5 μm. To test for nucleic acid content, the microparticles are dissolved in 0.1 M NaOH at 100° C. for 10 minutes. The A₂₆₀ is measured, and the amount of nucleic acid calculated from a standard curve. Incorporation of nucleic acid into microparticles is in the range of 1.76 g to 2.7 g nucleic acid per milligram PLG

Microparticles containing about 1 to 100 μg of nucleic acid are suspended in about 0.1 to 1 ml of 0.1 M sodium bicarbonate, pH 8.5, and orally administered to mice or humans. Regardless of the route of administration, an adjuvant can be administered before, during, or after administration of the nucleic acid. An adjuvant can increase the uptake of the nucleic acid into the cells, increase the expression of the antigen from the nucleic acid within the cell, induce antigen presenting cells to infiltrate the region of tissue where the antigen is being expressed, or increase the antigen-specific response provided by lymphocytes.

Evaluating Vaccine Efficacy

Before administering the vaccines described herein to humans, efficacy testing can be conducted using animals. In an example of efficacy testing, mice are vaccinated by intramuscular injection. After the initial vaccination or after optional booster vaccinations, the mice (and negative controls) are monitored for indications of vaccine-induced, SARS-specific immune responses. Methods of measuring immune responses are described in Townsend et al., J Virol, 71:3365-3374 (1997); Kuhober et al., J Immunol, 156: 3687-3695 (1996); Kuhrober et al., Int Immunol, 9:1203-1212 (1997); Geissler et al., Gastroenterology, 112:1307-1320 (1997); and Sallberg et al., J Virol, 71:5295-5303 (1997).

Anti-SARS serum antibody levels in vaccinated animals can be determined by known methods. The concentrations of antibodies can be standardized against a readily available reference standard.

Cytotoxicity assays can be performed as follows. Spleen cells from immunized mice are suspended in complete MEM with 10% fetal calf serum and 5×10⁻⁵ M 2-mercapto-ethanol. Cytotoxic effector lymphocyte populations are harvested after 5 days of culture, and a 5-hour ⁵¹Cr release assay is performed in a 96-well round-bottom plate using target cells. The effector to target cell ratio is varied. Percent lysis is defined as (experimental release minus spontaneous release)/(maximum release minus spontaneous release)×100.

Antibodies

This invention provides, inter alia, antibodies, or antigen-binding fragments thereof, to a SARS S, M, E, or N protein and/or specific fragments of the S, M, E, or N proteins, e.g., of the extracellular portion of the S protein.

Many types of anti-SARS protein antibodies, or antigen-binding fragments thereof, are useful in the methods of this invention. The antibodies can be of the various isotypes, including: IgG (e.g., IgG1, IgG2, IgG3, IgG4), IgM, IgA1, IgA2, IgD, or IgE. Preferably, the antibody is an IgG isotype, e.g., IgG1. The antibody molecules can be full-length (e.g., an IgG1 or IgG4 antibody) or can include only an antigen-binding fragment (e.g., a Fab, F(ab)₂, Fv or a single chain Fv fragment). These include monoclonal antibodies, recombinant antibodies, chimeric antibodies, human antibodies, and humanized antibodies, as well as antigen-binding fragments of the foregoing.

Monoclonal antibodies can be used in the new methods described herein. Monoclonal antibodies can be produced by a variety of techniques, including conventional monoclonal antibody methodology, e.g., the standard somatic cell hybridization technique of Kohler and Milstein, Nature 256: 495 (1975). Polyclonal antibodies can be produced by immunization of animal or human subjects. The advantages of polyclonal antibodies include the broad antigen specificity against a particular pathogen. See generally, Harlow, E. and Lane, D. (1988) Antibodies: A Laboratory Manual, Cold Spring Harbor Laboratory Press, Cold Spring Harbor, N.Y.

Useful immunogens for uses described herein include the SARS proteins described herein, e.g., SARS proteins expressed from optimized nucleic acid sequences.

Anti-SARS protein antibodies or fragments thereof useful in methods described herein may also be recombinant antibodies produced by host cells transformed with DNA encoding immunoglobulin light and heavy chains of a desired antibody. Recombinant antibodies may be produced by known genetic engineering techniques. For example, recombinant antibodies may be produced by cloning a nucleotide sequence, e.g., a cDNA or genomic DNA, encoding the immunoglobulin light and heavy chains of the desired antibody. The nucleotide sequence encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector. Prokaryotic or eukaryotic host cells may be used.

Expression in eukaryotic host cells is preferred because such cells are more likely than prokaryotic cells to assemble and secrete a properly folded and immunologically active antibody. However, any antibody produced that is inactive due to improper folding may be renatured according to well known methods (Kim and Baldwin, “Specific Intermediates in the Folding Reactions of Small Proteins and the Mechanism of Protein Folding,” Ann. Rev. Biochem., 51, pp. 459-89 (1982)). It is possible that the host cells will produce portions of intact antibodies, such as light chain dimers or heavy chain dimers, which also are antibody homologs.

It will be understood that variations on the above procedure are useful. For example, it may be desired to transform a host cell with DNA encoding either the light chain or the heavy chain (but not both) of an antibody. Recombinant DNA technology may also be used to remove some or all of the DNA encoding either or both of the light and heavy chains that is not necessary for binding, e.g., the constant region may be modified by, for example, deleting specific amino acids. The molecules expressed from such truncated DNA molecules are useful in the methods described herein. In addition, bifunctional antibodies may be produced in which one heavy and one light chain are anti-SARS protein antibody and the other heavy and light chain are specific for an antigen other than the SARS protein, or another epitope of the same protein, or of another SARS protein.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science, 240:1041-1043); Liu et al. (1987) PNAS, 84:3439-3443; Liu et al., 1987, J. Immunol., 139:3521-3526; Sun et al., (1987) PNAS, 84:214-218; Nishimura et al., 1987, Canc. Res., 47:999-1005; Wood et al., (1985) Nature, 314:446-449; and Shaw et al., 1988, J. Natl Cancer Inst., 80:1553-1559).

An antibody or an immunoglobulin chain can be humanized by methods known in the art. For example, once murine antibodies are obtained, variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol., 196:901-917, which are incorporated herein by reference). The light and heavy chain variable regions can, optionally, be ligated to corresponding constant regions.

Murine antibodies can be sequenced using art-recognized techniques. Humanized or CDR-grafted antibody molecules or immunoglobulins can be produced by CDR-grafting or CDR substitution, wherein one, two, or all CDRs of an immunoglobulin chain can be replaced. See e.g., U.S. Pat. No. 5,225,539; Jones et al., 1986, Nature, 321:552-525; Verhoeyan et al., 1988, Science, 239:1534; Beidler et al., 1988, J. Immunol., 141:4053-4060; and Winter, U.S. Pat. No. 5,225,539, the contents of all of which are hereby expressly incorporated by reference.

Winter describes a CDR-grafting method that may be used to prepare the humanized anti-SARS protein antibodies (UK Patent Application GB 2188638A, filed on Mar. 26, 1987; Winter U.S. Pat. No. 5,225,539), the contents of which is expressly incorporated by reference. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science, 229:1202-1207, by Oi et al., 1986, BioTechniques, 4:214, and by Queen et al. U.S. Pat. Nos. 5,585,089; 5,693,761; and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above. The recombinant DNA encoding the humanized antibody, or fragment thereof, can then be cloned into an appropriate expression vector.

Also included herein are humanized antibodies in which specific amino acids have been substituted, deleted, or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089 (e.g., columns 12-16), the contents of which are hereby incorporated by reference. The acceptor framework can be a mature human antibody framework sequence or a consensus sequence.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence. Other techniques for humanizing antibodies are described in Padlan et al. EP 519596 A1, published on Dec. 23, 1992.

Also within provided herein are antibodies that are produced in mice that bear transgenes encoding one or more fragments of an immunoglobulin heavy or light chain. See, e.g., U.S. Patent Publication No. 20030138421. Also provided are antibodies that are fully human (100% human protein sequences) produced in transgenic mice in which mouse antibody gene expression is suppressed and effectively replaced with human antibody gene expression (such mice are available, e.g., from Medarex, Princeton, N.J.). See, e.g., U.S. Patent Publication No. 20030031667.

An antibody, or antigen-binding fragment thereof, can be derivatized or linked to another functional molecule (e.g., another peptide or protein). For example, a protein or antibody can be functionally linked (by chemical coupling, genetic fusion, noncovalent association or otherwise) to one or more other molecular entities, such as another antibody, a detectable agent, a cytotoxic agent, a pharmaceutical agent, and/or a protein or peptide that can mediate association with another molecule (such as a streptavidin core region or a polyhistidine tag).

One type of derivatized protein is produced by crosslinking two or more proteins (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which a protein can be derivatized (or labeled) to include fluorescent compounds, various enzymes, prosthetic groups, luminescent materials, bioluminescent materials, and radioactive materials. Exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, β-galactosidase, acetylcholinesterase, glucose oxidase and the like. When a protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A protein can also be derivatized with a prosthetic group (e.g., streptavidin/biotin and avidin/biotin). For example, an antibody can be derivatized with biotin, and detected through indirect measurement of avidin or streptavidin binding.

Labeled proteins and antibodies can be used, for example, diagnostically and/or experimentally in a number of contexts, including (i) to isolate a predetermined antigen by standard techniques, such as affinity chromatography or immunoprecipitation; (ii) to detect a predetermined antigen (e.g., a SARS virion, e.g., in a cellular lysate or a serum sample) in order to evaluate the abundance and pattern of expression of the protein; and (iii) to monitor protein levels in tissue as part of a clinical testing procedure, e.g., to determine the efficacy of a given treatment regimen.

An anti-SARS protein antibody or antigen-binding fragment thereof may be conjugated to another molecular entity, typically a label or a therapeutic (e.g., a cytotoxic or cytostatic) agent or moiety.

Radioactive isotopes can be used in diagnostic or therapeutic applications. Radioactive isotopes that can be coupled to proteins and antibodies include, but are not limited to α-, β-, or γ-emitters, or β- and γ-emitters.

Viral Assays

The proteins and antibodies described herein can be tested using tranfected cells and/or SARS-infected cells. Protocols have been developed to grow SARS-CoV in culture. These methods use growth of Vero E6 cells. Supernatants from these cultures can contain up to 10⁷ copies of viral RNA per mL (Drosten et al., N Engl J Med, 348(20):1967-76, 2003; Ksiazek et al., N Engl J Med, 348(20):1953-66, 2003). A plaque reduction assay can be used to measure infectious titers of viral stocks, using established techniques (Bonavia et al., J Virol, 77 (4): 2530-8, 2003).

Western blotting can be used to test reactivity of protein products with anti-Histidine tag and antiserum to SARS-CoV as a screening step to measure protein expression and reactivity with antibodies produced in natural human infection.

Pharmaceutical Compositions

In another aspect, compositions, e.g., pharmaceutically acceptable compositions, are provided which include a protein or an antibody molecule described herein, formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Useful compositions are in the form of injectable or infusible solutions. A useful mode of administration is parenteral (e.g., intravenous, subcutaneous, intraperitoneal, intramuscular). For example, the protein or antibody can be administered by intravenous infusion or injection. In another embodiment, the protein or antibody is administered by intramuscular or subcutaneous injection.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The proteins, antibodies, and antibody-fragments can be administered by a variety of methods known in the art, although for many therapeutic applications. As will be appreciated by the skilled artisan, the route and/or mode of administration will vary depending upon the desired results.

In certain embodiments, a protein, an antibody, or antibody portion may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or antibody portion is 0.1-100 mg/kg, e.g., 1-10 mg/kg. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. The exact dosage can vary depending on the route of administration. For intramuscular injection, the dose range can be 100 μg (microgram) to 10 mg (milligram) per injection. Multiple injections may be needed.

The pharmaceutical compositions described herein can include a “therapeutically effective amount” or a “prophylactically effective amount” of a protein, antibody, or antibody portion. A “therapeutically effective amount” refers to an amount effective, at dosages and for periods of time necessary, to achieve the desired therapeutic result. A therapeutically effective amount of a nucleic acid vaccine or antibody or antibody fragment varies according to factors such as the disease state, age, sex, and weight of the individual, and the ability of the antibody or antibody portion to elicit a desired response in the individual. A therapeutically effective amount is also one in which any toxic or detrimental effects of the pharmaceutical composition is outweighed by the therapeutically beneficial effects. The ability of a compound to inhibit a measurable parameter can be evaluated in an animal model system predictive of efficacy in humans. Alternatively, this property of a composition can be evaluated by examining the ability of the compound to modulate, such modulation in vitro by assays known to the skilled practitioner.

A “prophylactically effective amount” refers to an amount effective, at dosages and for periods of time necessary, to achieve the desired prophylactic result, i.e., protective immunity against a subsequent challenge by the SARS virus. Typically, since a prophylactic dose is used in subjects prior to or at an earlier stage of disease, the prophylactically effective amount will be less than the therapeutically effective amount. Also provided herein are kits including a SARS protein, and/or an anti-SARS protein antibody or antigen-binding fragment thereof. The kits can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the SARS protein or antibody for administration; pharmaceutically acceptable carriers; and devices or other materials for administration to a subject.

Instructions for use can include instructions for diagnostic applications of the nucleic acid sequence, proteins, or antibodies (or antigen-binding fragment thereof) to detect SARS, in vitro, e.g., in a sample, e.g., a biopsy or cells from a patient, or in vivo. The instructions can include instructions for therapeutic or prophylactic application including suggested dosages and/or modes of administration, e.g., in a patient with a respiratory disorder. Other instructions can include instructions on coupling of the antibody to a chelator, a label or a therapeutic agent, or for purification of a conjugated antibody, e.g., from unreacted conjugation components.

As discussed above, the kit can include a label, e.g., any of the labels described herein. As discussed above, the kit can include a therapeutic agent, e.g., a therapeutic agent described herein. The kit can include a reagent useful for chelating or otherwise coupling a label or therapeutic agent to the antibody, e.g., a reagent discussed herein. Additional coupling agents, e.g., an agent such as N-hydroxysuccinimide (NHS), can be supplied for coupling the chelator, to the antibody. In some applications the antibody will be reacted with other components, e.g., a chelator or a label or therapeutic agent, e.g., a radioisotope. In such cases the kit can include one or more of a reaction vessel to carry out the reaction or a separation device, e.g., a chromatographic column, for use in separating the finished product from starting materials or reaction intermediates.

The kit can further contain at least one additional reagent, such as a diagnostic or therapeutic agent, e.g., a diagnostic or therapeutic agent as described herein, and/or one or more additional anti-SARS protein antibodies (or fragments thereof), formulated as appropriate, in one or more separate pharmaceutical preparations.

Other kits can include optimized nucleic acids encoding SARS proteins or anti-SARS protein antibodies, and instructions for expression of the nucleic acids.

Therapeutic Uses of Proteins and Antibodies

The new nucleic acid vaccines, proteins, and antibodies described herein have in vitro and in vivo diagnostic, therapeutic, and prophylactic utilities. For example, the nucleic acid vaccines can be administered to cells in culture, e.g., in vitro or ex vivo, or in a subject, e.g., in vivo, to treat, prevent, and/or diagnose SARS.

As used herein, the term “subject” is intended to include human and non-human animals. The term “non-human animals” includes all vertebrates, e.g., mammals and non-mammals, such as non-human primates, chickens and other birds, mice, dogs, cats, pigs, cows, and horses.

The proteins and antibodies can be used on cells in culture, e.g., in vitro or ex vivo. For example, cells can be cultured in vitro in culture medium and the contacting step can be effected by adding the SARS protein or the anti-SARS protein antibody or fragment thereof, to the culture medium.

Methods of administering nucleic acid vaccines and antibody molecules are described above. Suitable dosages of the molecules used will depend on the age and weight of the subject and the particular drug used. The nucleic acid vaccines can be used to prevent a SARS infection by inducing a protective immunity in the inoculated subject, or to treat an existing SARS infection if improved cellular immune responses can be useful in controlling the viral infection. The antibody molecules can be used to reduce or alleviate an acute SARS infection.

In other embodiments, immunogenic compositions and vaccines that contain an immunogenically effective amount of a SARS protein, or fragments thereof, are provided. Immunogenic epitopes in a protein sequence can be identified according to methods known in the art, and proteins, or fragments containing those epitopes can be delivered by various means, in a vaccine composition. Suitable compositions can include, for example, lipopeptides (e.g., Vitiello et al., J. Clin. Invest., 95:341 (1995)), peptide compositions encapsulated in poly(DL-lactide-co-glycolide) (“PLG”) microspheres (see, e.g., Eldridge et al., Molec. Immunol., 28:287-94 (1991); Alonso et al., Vaccine, 12:299-306 (1994); Jones et al., Vaccine, 13:675-81 (1995)), peptide compositions contained in immune stimulating complexes (ISCOMS) (see, e.g., Takahashi et al., Nature, 344:873-75 (1990); Hu et al., Clin. Exp. Immunol., 113:235-43 (1998)), and multiple antigen peptide systems (MAPs) (see, e.g., Tam, Proc. Natl. Acad. Sci. U.S.A., 85:5409-13 (1988); Tam, J. Immunol. Methods, 196:17-32 (1996)). Toxin-targeted delivery technologies, also known as receptor-mediated targeting, such as those of Avant Immunotherapeutics, Inc. (Needham, Mass.) can also be used.

Useful carriers that can be used with immunogenic compositions and vaccines are well known, and include, for example, thyroglobulin, albumins such as human serum albumin, tetanus toxoid, polyamino acids such as poly L-lysine, poly L-glutamic acid, influenza, hepatitis B virus core protein, and the like. The compositions and vaccines can contain a physiologically tolerable (i.e., acceptable) diluent such as water, or saline, typically phosphate buffered saline. The compositions and vaccines also typically include an adjuvant. Adjuvants such as incomplete Freund's adjuvant, aluminum phosphate, aluminum hydroxide, or alum are examples of materials well known in the art. Additionally, CTL responses can be primed by conjugating SARS proteins (or fragments, derivatives or analogs thereof) to lipids, such as tripalmitoyl-S-glycerylcysteinyl-seryl-serine (P₃CSS).

Immunization with a composition or vaccine containing a protein composition, e.g., via injection, aerosol, oral, transdermal, transmucosal, intrapleural, intrathecal, or other suitable routes, induces the immune system of the host to respond to the composition or vaccine by producing large amounts of CTL's, and/or antibodies specific for the desired antigen. Consequently, the host typically becomes at least partially immune to later infection (e.g., with SARS-CoV), or at least partially resistant to developing an ongoing chronic infection, or derives at least some therapeutic benefit. In other words, the subject is protected against subsequent viral infection by the SARS virus.

Other Uses of Proteins and Antibodies

An anti-SARS protein antibody (e.g., monoclonal antibody) can be used to isolate SARS protein or SARS virions by standard techniques, such as affinity chromatography or immunoprecipitation. Moreover, an anti-SARS protein antibody can be used to detect a SARS protein (e.g., in a cellular lysate or cell supernatant or blood sample), e.g., to screen samples for the presence of SARS, or to evaluate the abundance and pattern of expression of SARS. Anti-SARS protein antibodies can be used diagnostically to monitor SARS protein or SARS levels in tissue as part of a clinical testing procedure, e.g., to, for example, determine the efficacy of a given treatment regimen.

SARS proteins, and fragments thereof can be used to detect expression of a SARS receptor, e.g., to identify cells and tissues susceptible to SARS infection, or to isolate a SARS receptor on a host cell.

### EXAMPLES

The invention is further described in the following examples, which do not limit the scope of the invention described in the claims.

### Example 1

### Construction of Codon-Optimized Coding Sequences of SARS Proteins

The native SARS-CoV S gene sequence shows a high AU-rich bias as compared to the codon usage preferred by mammalian genes. To generate DNA for efficient expression of the S protein and S protein fragments, codon-optimized nucleic acids were constructed. These codon-optimized nucleic acids were designed to express polypeptides with amino acid sequences identical to sequences encoded by the native SARS-CoV S protein but with codons known to be efficiently translated in mammalian host cells. Substitution of viral codons for mammalian codons can facilitate high levels of expression of viral proteins in recombinant systems.

The codon usage of published SARS-CoV S gene sequences (24, 35) was analyzed by the MacVector software (V. 7.2, Accelrys, San Diego, Calif.) against that of the Homo sapiens genome. Sequences were generated in which the codons in the S gene that are less optimal for mammalian expression were changed to the codons more preferred in mammalian systems. The sequences were also designed to avoid unwanted RNA motifs, such as internal TATA-boxes, chi-sites, ribosomal entry sites, AT-rich or GC-rich sequence stretches, repeat sequences, sequences likely to encode RNA with secondary structures, (cryptic) splice donor and acceptor sites, or branch points.

The following codon-optimized nucleic acids encoding fragments of the S gene were chemically synthesized: S1.1, encoding amino acids 12 to 535 of the S protein; S1.2, encoding amino acids 534 to 798 of the S protein; and S2, encoding amino acids 797 to 1255 of the S protein. Fragments were synthesized by Geneart (Regensburg, Germany). The nucleic acid encoding the S1.1 fragment was synthesized with cleavage sites for restriction enzymes NsiI and BamHI flanking the coding region. The nucleic acids encoding the S1.2 and S2 fragments were synthesized with PstI and BamHI sites flanking the coding portion. Addition of the restriction enzyme sites facilitated subcloning into DNA vectors.

Next, the codon-optimized S gene segments were individually subcloned into the DNA vaccine vector pSW3891(42) which is a modified form of the pJW4303 vector (20). The pSW3891 vector contains a cytomegalovirus immediate early promoter (CMV-IE) with its downstream Intron A sequence for initiating transcription of eukaryotic gene inserts and a bovine growth hormone (BGH) poly-adenylation signal for termination of transcription. For certain constructs, a human tissue plasminogen activator (tPA) leader sequence was included to direct expression of secreted proteins. The vector also contains the ColE1 origin of replication for prokaryotic replication and the kanamycin resistance gene for selective growth in antibiotic containing media.

Additional DNA plasmids encoding the full length S (aa 1-1255), soluble S.dTM (aa 12-1192), S1 (aa 12-798), and extracellular portion of S2.dTM (aa 797-1192) were further produced by ligating the codon-optimized fragments described above. Constructs for expression of the S protein and fragments listed in Table 1 were generated.

Each individual DNA plasmid was confirmed by DNA sequencing before large amounts of DNA plasmids were prepared from Escherichia coli (HB101 strain) with a Mega purification kit (Qiagen, Valencia, Calif.) for both in vitro transfection and in vivo animal immunization studies.

Codon-optimized sequences encoding the fragments of the SARS-CoV N protein, E protein, and M protein were constructed in the same manner as the S protein fragments. These are also listed in Table 1.

TABLE 1
Codon-optimized SARS-CoV Nucleic Acid/Amino Acid Sequences

| Name | Description |
|---|---|
| wt-S | Full-length S protein (amino acids 1-1255) |
| S1 | S protein amino acids 12-798 |
| tPA-S2 | S protein amino acids 797-1255 with N-terminal tPA leader |
| | sequence |
| S1.1 | S protein amino acids 12-535 |
| tPA-S1.2 | S protein amino acids 534-798 with N-terminal tPA |
| | leader sequence |
| S.dTM | S protein extracellular domain (amino acids 1-1192) |
| S2.dTM | S2 protein fragment extracellular domain (amino |
| | acids 797-1192) |
| tPA-S1 | S1 fragment with N-terminal tPA leader sequence |
| tPA-S2 | S2 fragment with N-terminal tPA leader sequence |
| tPA-S.dTM | S protein lacking the transmembrane domain (amino |
| | acids 12-1192) with N-terminal tPA leader sequence |
| tPA-S1.1 | N-terminal tPA leader sequence + S1.1 fragment |
| tPA-S1.2 | N-terminal tPA leader sequence + S1.2 fragment |
| E (1-77) | amino acids 1-77 of the envelope protein |
| M (1-222) | amino acids 1-222 of the membrane protein |
| N (1-424) | amino acids 1-424 of the nucleocapsid protein |
| tPA-E | N-terminal tPA leader sequence + E amino acid sequence |
| tPA-M | N-terminal tPA leader sequence + M amino acid sequence |

### Example 2

### Antibody Responses in DNA-immunized Rabbits

Immunization. NZW Rabbits (female, ˜2 kg each) were purchased from Millbrook Farms (Millbrook, Mass.) and housed in the Department of Animal Medicine at the University of Massachusetts Medical School (UMMS) in accordance with IACUC approved protocols. The animals were immunized with a Helios gene gun (Bio-Rad, Hercules, Calif.) at the shaved abdominal skin as previously reported (43). A total of 36 μg of plasmid DNA was administrated to each individual rabbit for each immunization at weeks 0, 2, 4 and 8. Serum samples were taken prior to the first immunization and 2 weeks after each immunization for analyses of S-specific antibody responses.

ELISA to Determine Anti-S IgG Responses. ELISA assays were conducted to measure the anti-S IgG responses in immunized rabbits. Flat-bottom 96-well plates were coated with 100 μl of ConA (50 μg/ml) for 1 hour at room temperature, and washed 5 times with PBS containing 0.1% Triton X-100. Subsequently, the plates were incubated overnight at 4° C. with 100 μl of transiently expressed SARS-CoV S antigen at 1 μg/ml. Coating antigens were isolated from 293T cells transiently transfected with the tPA-S.dTM and tPA-S1.2 constructs. Plates were washed five times as above and blocked with 200 μl/well of blocking buffer (5% non-fat dry milk, 4% whey, 0.5% Tween-20 in PBS at pH 7.2) for 1 hour. After five washes, 100 μl of serially diluted rabbit serum was added in duplicate wells and incubated for 1 hour. After another set of washes, the plates were incubated for 1 hour at room temperature with 100 μl of biotinylated anti-rabbit IgG (Vector Laboratories, Burlingame, Calif.) diluted at 1:1000 in Whey dilution buffer (4% Whey, 0.5% Tween-20 in PBS). Then 100 μl of horseradish peroxidase-conjugated streptavidin (Vector Laboratories) diluted at 1:2000 in Whey buffer was added to each well and incubated for 1 hour. After the final wash, the plates were developed with 3,3′,5,5′ Tetramethybenzidine solution at 100 μl per well (Sigma, St. Louis, Mo.) for 3.5 minutes. The reactions were stopped by adding 25 μl of2 M H₂SO₄, and the plates were read at OD 450 nm.

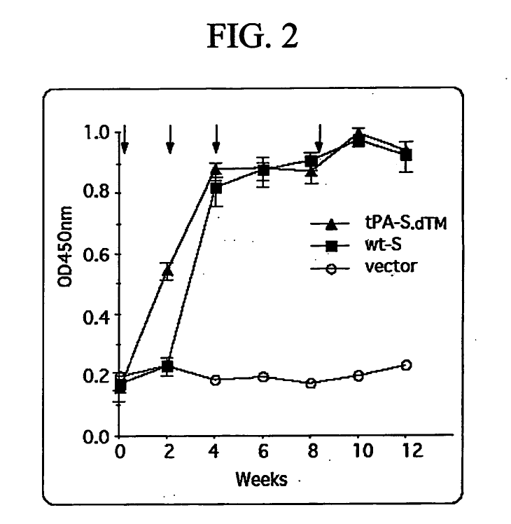

Results. The codon-optimized DNA constructs encoding wt-S and tPA-S.dTM induced robust anti-S IgG responses in immunized NZW rabbits FIG. 2. The tPA-S.dTM construct induced positive anti-S antibody responses after a single immunization. The wt-S vaccine induced a detectable response after two immunizations. The antibody responses to both vaccines peaked within four immunizations.

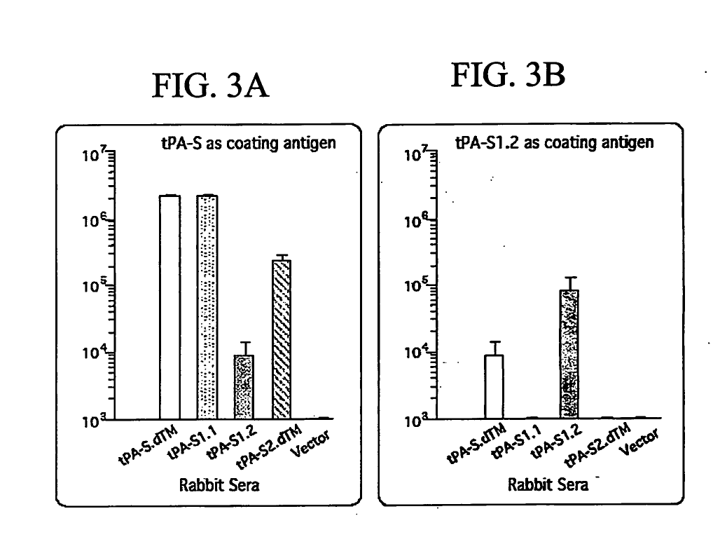

Codon-optimized DNA constructs expressing other segments of the S protein also induced significant anti-S antibody responses FIG. 3. First, antisera induced by tPA-S.dTM, tPA-S1.1, tPA-S1.2 and tPA-S2.dTM constructs were tested in parallel for reactivity to full length S protein by ELISA. Antisera were collected from animals that had been immunized with the DNA constructs four times. In these assays, the titers of tPA-S-reactive antibodies induced by tPA-S1.2 and tPA-S2.dTM constructs were lower than the titers induced by tPA-S.dTM or TPA-S1.1 (FIG. 3A).

Next, antisera induced by tPA-S.dTM, tPA-S1.1, tpA-S1.2 and tPA-S2.dTM constructs were tested for reactivity to the S1.2 antigen. In these assays, high titers of antibody induced by tPA-S.dTM and tPA-S1.2 and tPA-S2.dTM constructs were detected. As expected, sera raised against the tPA-S1.1 and tPA-S2 constructs (which do not contain the S1.2 fragment) did not show detectable reactivity to the S1.2 fragment. These data suggest that the S1.2 fragment is immunogenic, but that the S1.2 fragment within the full length S protein may have poor surface accessibility. The observation that sera induced by tPA-S.dTM was less effective in recognizing the S1.2 antigen than the S antigen implies that a large portion of the antibody response to the protein expressed by this construct is directed at the N-terminal S1.1 and C terminal S2 segments.

### Example 3

### Domain-specific Anti-S Antibody Responses Induced by DNA Immunization

The specificity of rabbit sera induced by the S protein-encoding DNA constructs was further analyzed by Western Blot.

Western blot analysis of in vitro expressed S antigens. Codon optimized DNA constructs encoding various fragments of the S protein were first transfected into the human embryonic kidney 293T cells using calcium phosphate precipitation method. Briefly, 2×10⁶ 293T cells (50% confluent) in a 60 mm dish were transfected with 10 μg of plasmid DNA and were harvested 72 hours later. After heat treatment at 90° C. for 5 minutes in loading buffer (50 mM Tris.HCl, pH 6.8, 100 mM dithiothreitol, 2% SDS, 0.1% bromophenol blue, 10% glycerol), equal amounts of transiently expressed S antigens (10 ng of protein per lane) were subjected to SDS-polyacryamide gel electrophoresis (SDS-PAGE), transferred onto PVDF membranes (Bio-Rad), and blocked overnight at 4° C. in blocking buffer (0.2% I-block, 0.1% Tween-20 in 1×PBS). Membranes were incubated with a 1:200 dilution of rabbit sera immunized with the specified DNA construct. Membranes were washed and incubated with alkaline phosphatase-conjugated goat anti-rabbit IgG at a 1:5000 dilution. Signals were detected using a chemiluminescence Western-Light Kit (Tropix, Bedford, Mass.). As specified in the results section, some of the transfected samples were prepared in the presence of 4 M urea in the loading buffer to ensure complete denaturation before SDS-PAGE.

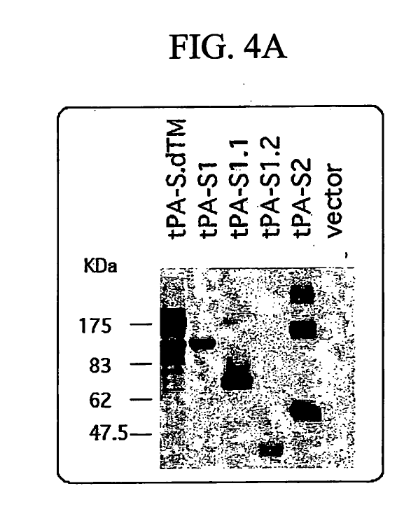

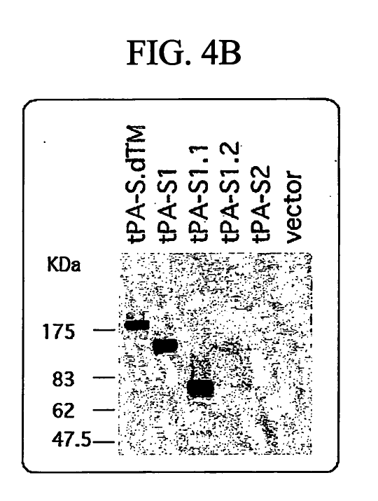

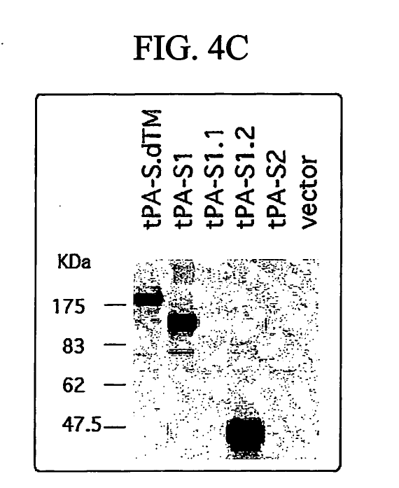

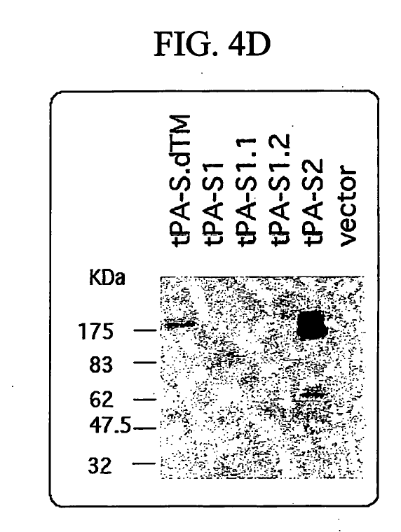

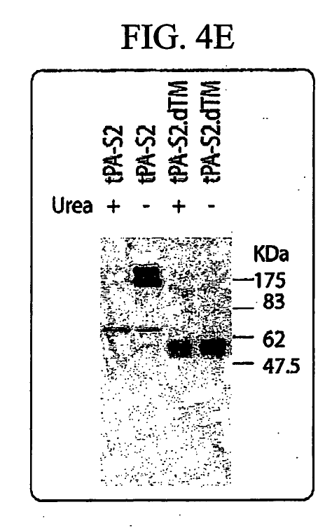

Results. Antisera from rabbits immunized with the tPA-S.dTM DNA construct recognized the full length S and each of the S segments (S1, S1.1, S1.2 and S2) (FIG. 4A). The tPA-S1.1 DNA construct elicited antibody responses recognizing the autologous S1.1 antigen as well as the full length S and S1 antigens which contain the S1.1 segment, but not the S1.2 or S2 segments (FIG. 4B). Similarly, the tPA-S1.2 DNA construct induced antibodies recognizing the autologous S1.2 and the two larger S antigens (full length S and S1), but not the non-overlapping S1.1 or S2 segments (FIG. 4C). Finally, the tPA-S2.dTM DNA construct induced antibody responses recognizing its autologous S2 segment and, to a lesser degree, the full length S protein, but not any of the other unrelated S1, S1.1 or S1.2 segments (FIG. 4D). These data confirm that the DNA constructs encoding segments of the S protein induce antibodies specific for each segment. Segment-specific antibodies were used to map the potential neutralizing domains of the S protein.

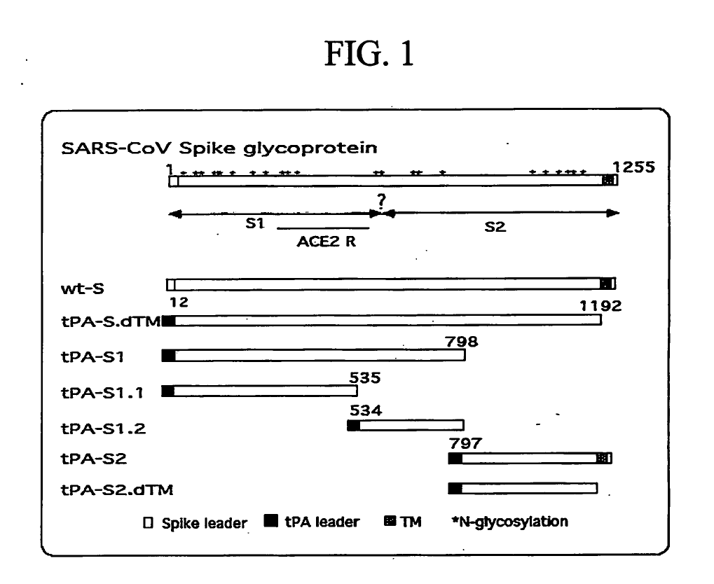

These experiments also demonstrated that the C-terminal TM region of S protein plays an important role in the oligomerization of S protein. As described above, two codon-optimized constructs expressing S2 were generated: tPA-S2, which encodes an S2 segment including the TM domain; and tPA-S2.dTM, which encodes an S2 segment lacking the TM domain (FIG. 1). As shown in FIGS. 4A and 4D, three bands were detected in the lane containing S2. These bands most likely represent a monomer, trimer, and a higher molecular weight complex based on their apparent molecular weights of approximately 50 KDa, 150 KDa (for the two faster-migrating bands). The potential of S2 to form heat-resistant oligomers was further confirmed by an additional experiment in which S antigens were mixed with 4M urea before loading onto SDS-PAGE to dissociate the oligomer structure (FIG. 4E). Antisera from animals immunized with the tPA-S2.dTM construct was used for detection in this experiment. This experiment showed that the S2 antigen, but not S2-dTM, formed stable oligomers which were present in the conventional denaturing SDS-PAGE but sensitive to urea treatment.

### Example 4

### Sera Induced by S-expressing DNA Constructs Recognizes Spike Proteins Associated with SARS-CoV Virions

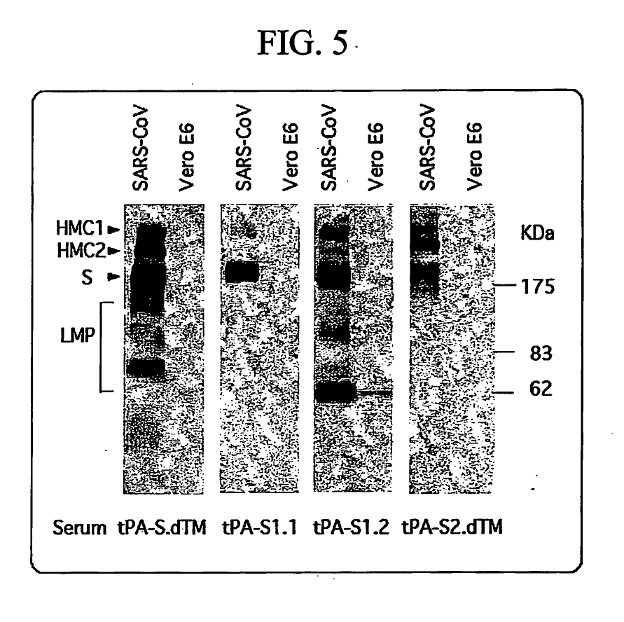

The ability of sera from mice immunized with DNA to recognize virus associated SARS-CoV S protein was analyzed. Preparations of SARS-CoV were lysed, subjected to SDS-PAGE, and transferred to PVDF membranes for Western blotting. Rabbit antisera from animals immunized with DNA constructs expressing either full length S protein or segments of the S protein recognized a dominant band around 190 KDa (indicated by arrow S), the expected position of the SARS-CoV S protein (FIG. 5, lanes 1, 3, 5). By comparing the additional S protein bands detected by different S segment specific rabbit sera, our data also demonstrated the possibility of spontaneous proteocleavage on the S protein leading to several smaller low molecular weight products (LMP) which were mainly detected by the full length S, S1.1 and S1.2 sera (FIG. 5, lanes 1, 3, 5), but not by S2 sera (FIG. 5, lane 7). Two major high molecular weight complexes (HMC1 and HMC2) were detected by the antisera. The HMC2 band was detected by the fill length S and the S2 sera but not effectively by the S1.1 or S1.2 sera. The other high molecular complex, HMC1, was recognized by the S, S1.1 and S1.2 sera and to a less extent by the S2 serum. The HMC1 may correspond to an oligomer of full-length of S and HMC2 may correspond to an oligomer of cleaved S2 fragments.

### Example 5

### Neutralization of SARSCoV by Antisera from Rabbits Immunized with Codon-Optimized DNA Constructs

The ability of anti-S specific antibodies in DNA immunized rabbit sera was further tested by two neutralization assays for their ability to neutralize SARS-CoV cultured in VeroE6 cells.

Production of SARS-Co V viral stocks. A stock of the SARS-CoV Urbani strain was obtained from U.S. Center for Diseases Control and Prevention (Atlanta, Ga.). For propagation of the SARS-CoV viral stock, Vero E6 cells (2×10⁶ cells) were infected with a multiplicity of infection (MOI) of 0.01 and cultured for 3-4 days at 37° C./5% CO₂. The culture supernatant was harvested at the onset of cytopathic effect (CPE) and filtered through a 0.45 μm membrane to remove the cell debris. The TCID₅₀ of viral stock was measured in 96-well flat bottom plates. To inactivate the virus for ELISA and Western blot analysis, the virus stocks were treated with 1% Triton-X 100 in TBS (Tris-buffered saline, pH 7.6) for 1 hour at 4° C. Inactivation of SARS-CoV was confirmed using a Standard Operational Procedure (SOP) approved by the Institutional Biosafety Committee at the University of Massachusetts Medical School.

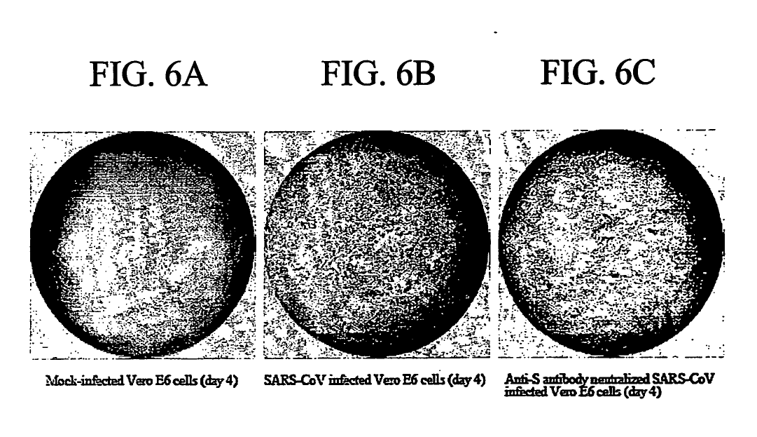

CPE assays. CPE was observed daily to follow the conditions of virus infected cells cultured in the presence or absence of sera from DNA-immunized rabbits. Sample CPE pictures are shown in FIGS. 6A-6C. FIG. 6A shows a plate of mock-infected Vero E6 cells after 4 days of culture. FIG. 6B shows a plate of SARS-CoV infected Vero E6 cells four days after infection. FIG. 6C shows a plate of SARS-CoV infected Vero E6 cells cultured in the presence of anti-S antibody, four days after infection. These pictures show that the mock-infected cells and infected cells cultured with anti-S antibody appear to be smooth and translucent, whereas the cells infected with SARS-CoV appear to be small, rounded, less translucent, and the plate is patchy with gaps where cells have detached. Thus, the anti-S antisera protect Vero E6 cells from the cytopathic effects of SARS-CoV infection.

In vitro neutralization assays. SARS-CoV neutralization assays were performed with triplicate testing wells in 96-well flat bottom plates in a biosafety level-3 (BL-3) laboratory. For the initial step of the assays, 400 TCID₅₀ of virus in 50 μl/well was incubated with 50 μl of serially diluted rabbit sera or tissue culture medium for 1 hour at 37° C. After incubation, 100 μl of Vero E6 cells (20,000 cells) was added to each well. The neutralization antibody against SARS-CoV was measured by two different assays. In the first neutralization assay, results were measured by cytopathic effect (CPE) on day 4 of infection, which was observed under a microscope. The neutralizing antibody titer was defined as the reciprocal of the highest serum dilution at which no CPE breakthrough in any of the triplicate testing wells was observed.

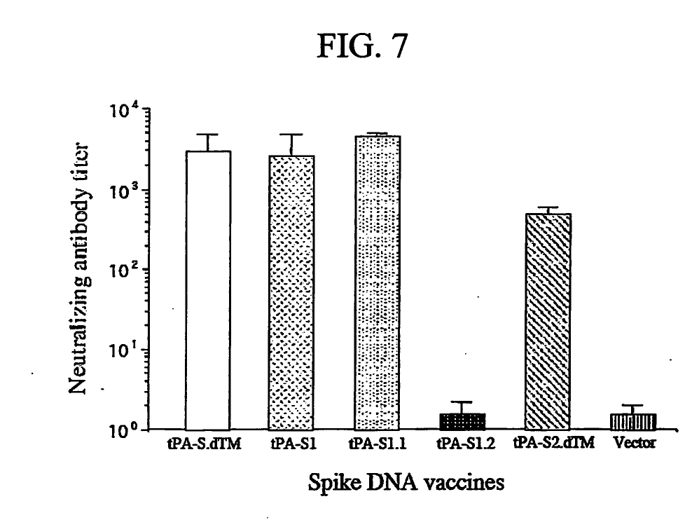

The results of assays to determine neutralizing titers based on CPE are summarized in FIG. 7. The neutralizing antibody titers are presented as the geometric means of the highest antibody dilutions that could still completely block the CPE in triplicate wells. The full length S, S1 and S1.1 DNA constructs elicited strong neutralizing antibody responses. The S2 DNA construct also elicited positive neutralizing antibody responses but at a lower level. The S1.2 DNA construct did not elicit meaningful neutralizing antibody responses against the SARS-CoV, same as the vector control rabbit sera.

The second assay in vitro neutralization assay used neutral red staining of live cells to identify the percentage of Vero E6 cells surviving SARS-CoV infection in the presence of anti-S antibody. Five days after infection, when more than 70% cells formed CPE in the viral control wells, culture medium was removed from the testing wells and 100 μl of 10% neutral red in DMEM medium was added to each well. After incubation for 1 hour at 37° C., the neutral red medium was removed, the plates Were washed twice with PBS (pH 7.2) and 100 μl of acid alcohol (1% acetic acid in 50% ethanol) was added to each well. After incubation for 30 minutes at room temperature, the absorbance was read at A₅₄₀. Percent neutralization at a given serum dilution was determined by calculating the difference in absorption (A₅₄₀) between test wells (cells, serum sample, and virus) and virus control wells (cells and virus) and dividing this result by the difference in absorption between cell control wells (cells only) and virus control wells (26). In our assay system, sera were considered positive for neutralizing antibody activities when the titers were above 50% inhibition as compared with the virus controls.

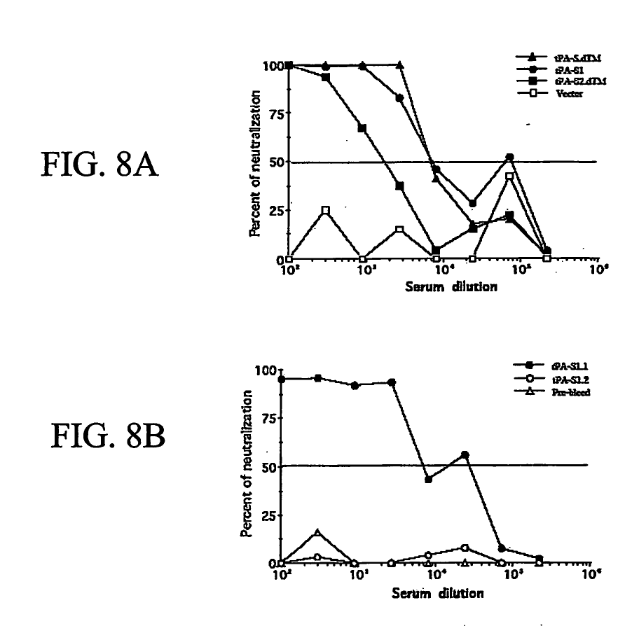

The neutralizing titers in the neutral red assay are expressed as the highest sera dilutions that inhibited infection by 50% (FIG. 8). Similar to the CPE assay, the S, S1 and S2 DNA constructs elicited neutralizing antibody responses (FIG. 8A) as well as the S1.1 DNA construct (FIG. 8B). The S1.2 DNA construct was ineffective in inducing antibodies capable of neutralizing SARS-CoV infection in this assay.

These data suggest that there is more than one neutralizing domain in either the N-terminal S1.1 or the C-terminal S2 segments, but not in the middle S1.2 segment. The neutralizing antibody titers in both CPE and neutral red assays are summarized in Table 2. Overall, the titers in neutral red assay (50% neutralization) were higher than those in CPE assay (100% neutralization) reflecting the more stringent criteria of the CPE assay.

TABLE 2
SARS-CoV Neutralizing Antibody Titers in Rabbit Sera
Immunized with Different S Protein DNA Constructs

| Vaccine | CPE assay | Neutral red assay |
|---|---|---|
| groups | (100% neutralization) | (50% neutralization) |
| tPA-S.dTM | 2938.49 | 4669.16 |
| tPA-S1 | 2561.44 | 5486.36 |
| tPA-S2.dTM | 492.95 | 878.63 |
| tPA-S1.1 | 4436.55 | 8843.93 |
| tPA-S1.2 | <30 | <30 |
| Vector | <30 | <30 |
| Pre-immune | <30 | <30 |

The values are the geomatric means from 4 independent assays by using rabbit sera from two animals per group.

### Example 6

### The S Protein of SARS-CoV is Glycosylated

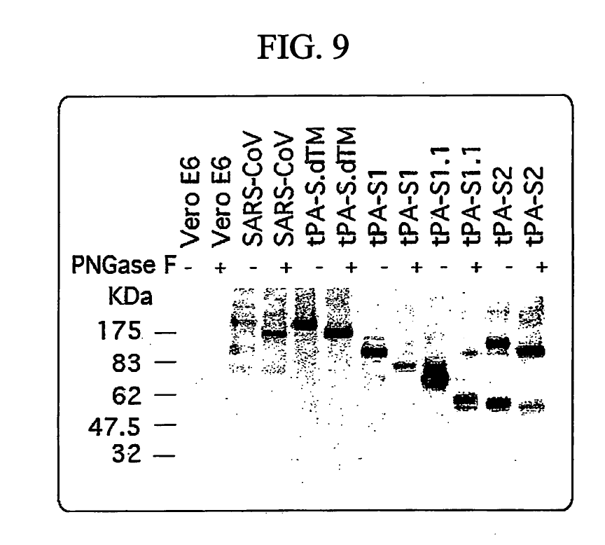

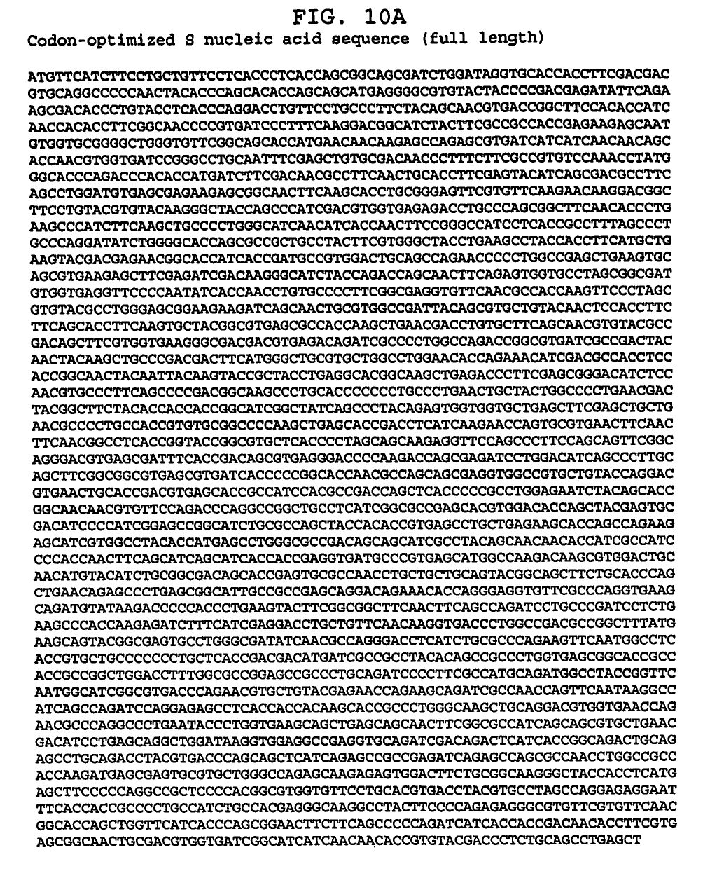

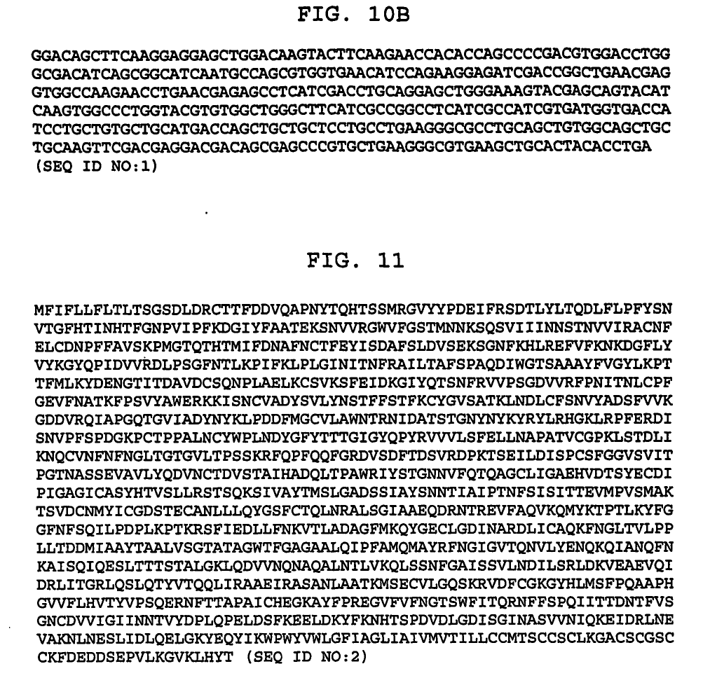

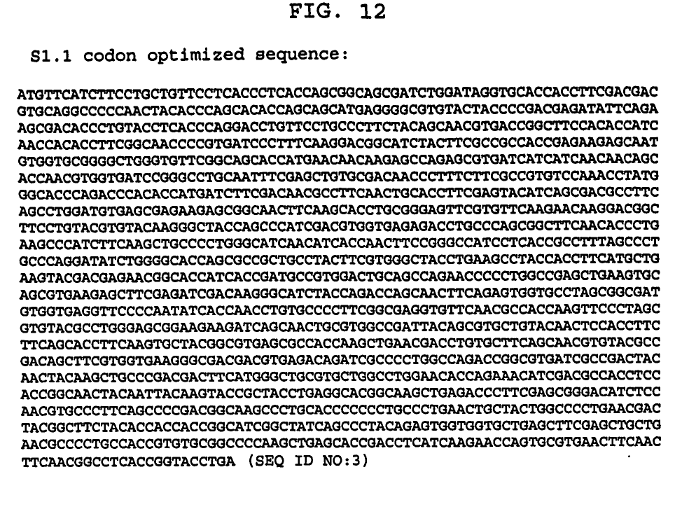

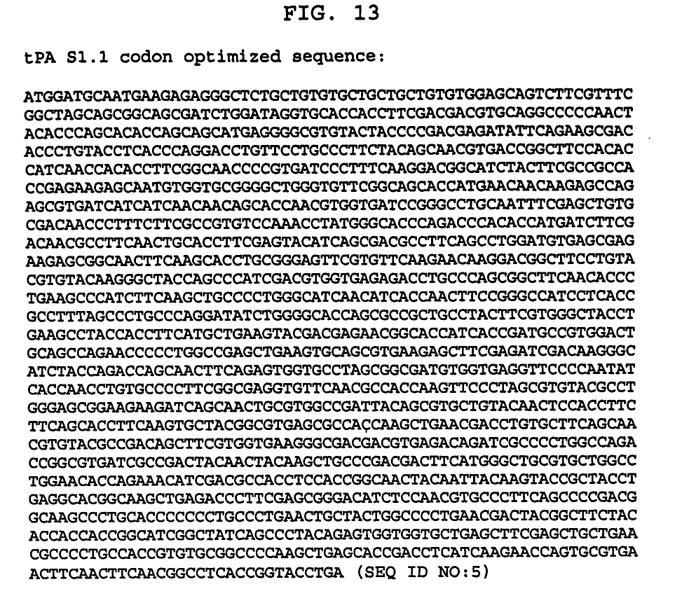

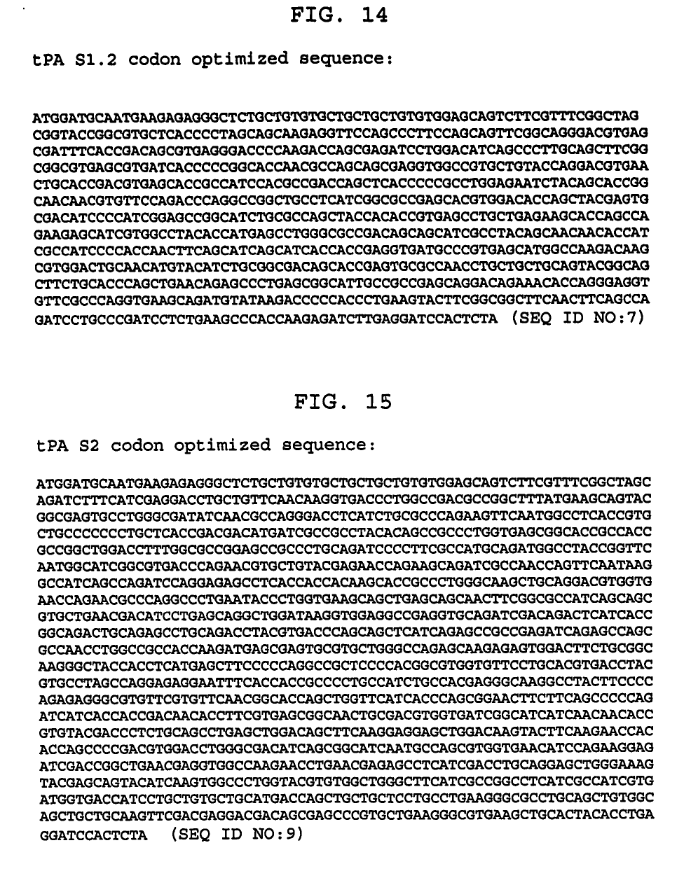

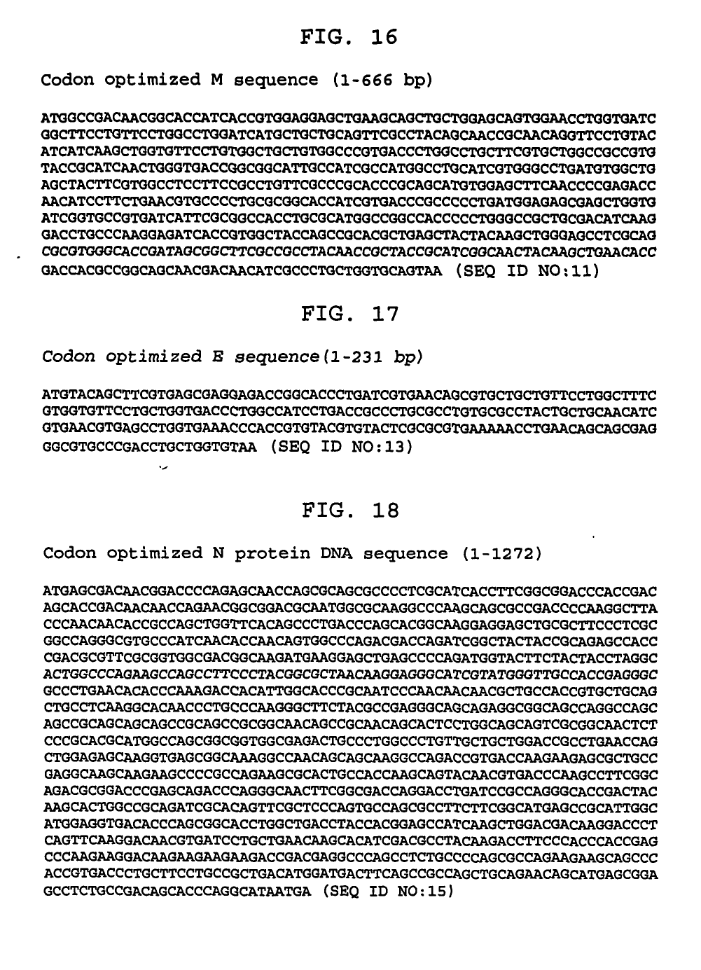

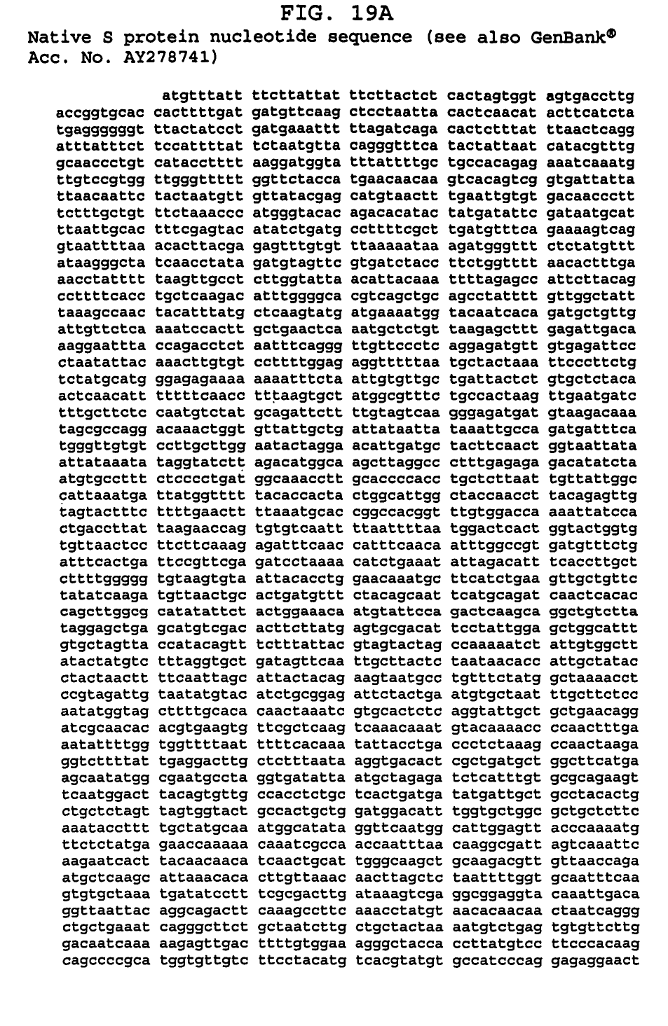

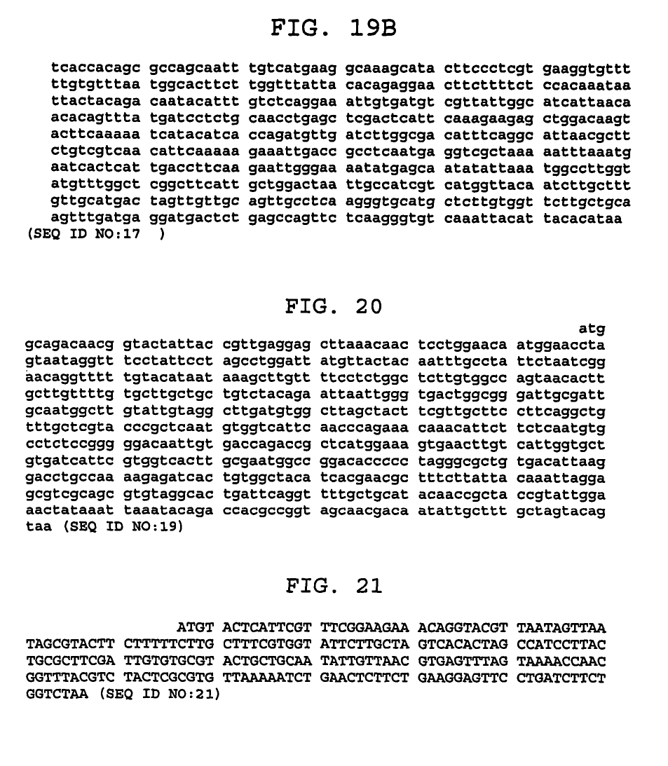

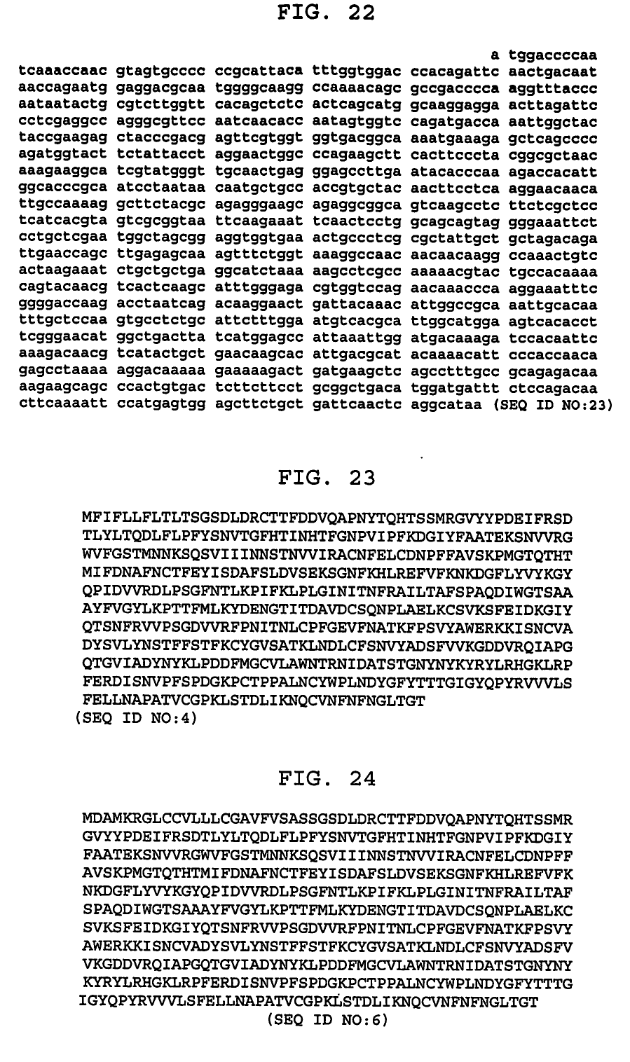

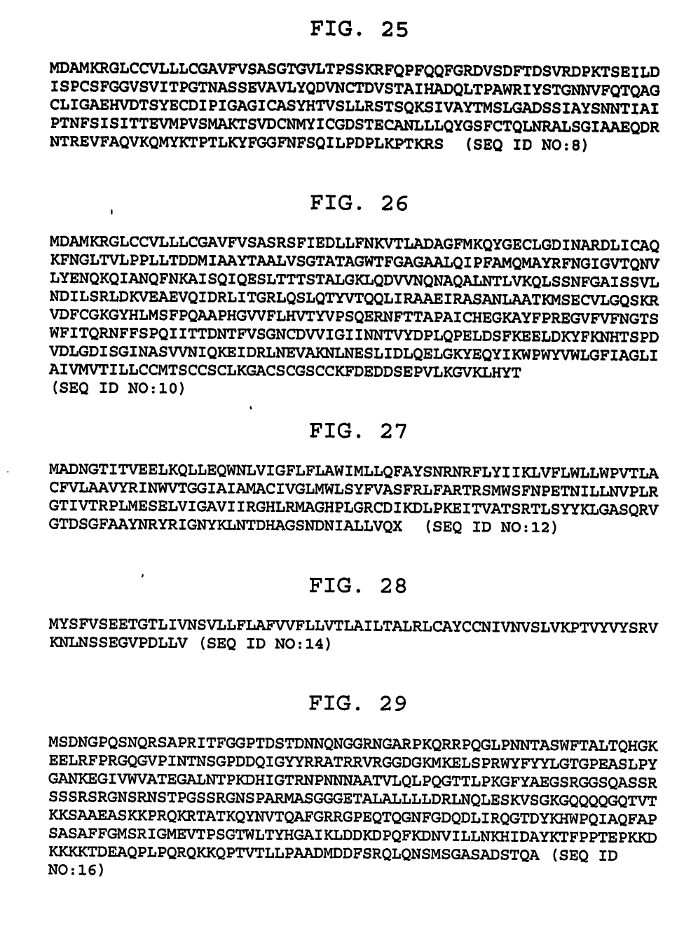

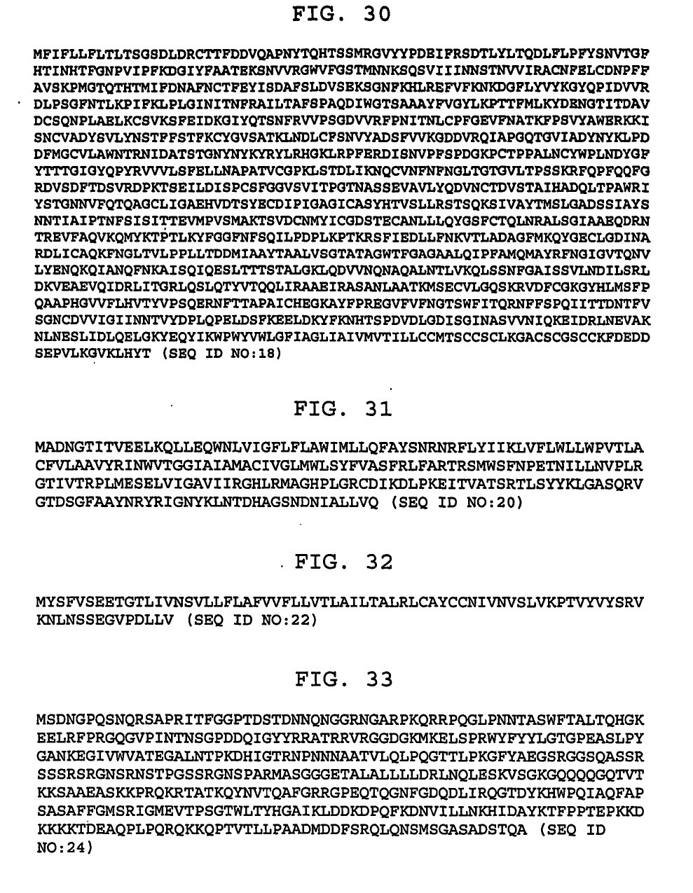

The S protein has 23 potential N-glycosylation sites throughout its entire sequence. Most of these sites are predicted to be surface exposed and extensively glycosylated to act as attachment proteins. Indeed, the full-length S protein as well as the fragments of the S protein migrate on SDS-PAGE at positions significantly higher than the theoretical molecular weights estimated from the number of amino acid residues in the polypeptides. To investigate N-glycosylation in the S protein, different forms of the S protein from transiently transfected 293T cells were treated with PNGaseF to remove the N-glycans. PNGaseF is an amidase which cleaves between the innermost GlcNAc and asparagines residues of high mannose, hybride and complex oligosaccharides from N-linked glycoprotein (23, 41). Notably, the full length S protein, S1.1, S1.2 and S1 displayed reduced molecular weight by SDS-PAGE after PNGase F treatment (FIG. 9). The mobility shift in molecular weights after deglycosylation was consistent with the expected molecular weights from the core amino acid sequences of each polypeptide without any glycosylations. This demonstrates that the S proteins produced in 293T cells are glycosylated in a manner similar to that predicted by the presence of N-glycan sites (24, 35).

We also examined the S protein on the viral particles of SARS-CoV grown from the cultured Vero E6, and found that the S protein was N-glycosylated. After treatment with PNGaseF, the molecular weight of S protein associated with the SARS-CoV virons was reduced to a degree similar to the degree seen with S protein produced from the transiently transfected 293T cells (FIG. 9).

### REFERENCES CITED

1. Bosch et al., 2003, The coronavirus spike protein is a class I virus fusion protein: structural and functional characterization of the fusion core complex, J Virol, 77:8801-11.
2. Callow et al., 1990, The time course of the immune response to experimental coronavirus infection of man, Epidemiol Infect, 105:435-46.
3. Chapman et al., 1991, Effect of intron A from human cytomegalovirus (Towne) immediate-early gene on heterologous expression in mammalian cells, Nucleic Acids Res, 19:3979-86.
4. Corbet et al., 2000, Construction, biological activity, and immunogenicity of synthetic envelope DNA vaccines based on a primary, CCR5-tropic, early HIV type 1 isolate (BX08) with human codons, AIDS Res Hum Retroviruses, 16:1997-2008.
5. de Arriba et al., 2002, Mucosal and systemic isotype-specific antibody responses and protection in conventional pigs exposed to virulent or attenuated porcine epidemic diarrhoea virus, Vet Immunol Immunopathol, 85:85-97.
6. Frana et al., 1985, Proteolytic cleavage of the E2 glycoprotein of murine coronavirus: host-dependent differences in proteolytic cleavage and cell fusion, J Virol, 56:912-20.
7. Gallagher, T. M, 1996, Murine coronavirus membrane fusion is blocked by modification of thiols buried within the spike protein, J Virol, 70:4683-90.
8. Gallagher, T. M., and M. J. Buchmeier, 2001, Coronavirus spike proteins in viral entry and pathogenesis, Virology, 279:371-4.
9. Holmes, K, 2001, Coronaviruses, p. 1187-1203, In D. Knipe, P. Howley, D. Griffin, R. Lamb, M. Martin, B. Roizman, and S. Straus (ed.), Fields Viology, 4 ed, vol. 1. Lippincott Williams & Wilkins.
10. Jackwood et al., 2001, Spike glycoprotein cleavage recognition site analysis of infectious bronchitis virus, Avian Dis, 45:366-72.
11. Koo et al., 1999, Protective immunity against murine hepatitis virus (MHV) induced by intranasal or subcutaneous administration of hybrids of tobacco mosaic virus that carries an MHV epitope, Proc Natl Acad Sci U S A, 96:7774-9.
12. Kraaijeveld et al., 1980, Enzyme-linked immunosorbent assay for detection of antibody in volunteers experimentally infected with human coronavirus strain 229, E. J Clin Microbiol, 12:493-7.
13. Krokhin et al., 2003, Mass Spectrometric Characterization of Proteins from the SARS Virus: A Preliminary Report, Mol Cell Proteomics, 2:346-56.
14. Ksiazek et al., 2003, A novel coronavirus associated with severe acute respiratory syndrome, N Engl J Med, 348:1953-66.
15. Lai, M. M., and K. Holmes, 2001, Coronarviridae: The Viruses and Their Replication, p. 1163-1185, In D. Knipe, P. Howley, D. Griffin, R. Lamb, M. Martin, B. Roizman, and S. Straus (ed.), Fields Viology, 4 ed, vol. 1. Lippincott Williams & Wilkins.
16. Leparc-Goffart et al., 1998, Targeted recombination within the spike gene of murine coronavirus mouse hepatitis virus-A59: Q159 is a determinant of hepatotropism, J Virol, 72:9628-36.
17. Lewicki, D. N., and T. M. Gallagher, 2002, Quaternary structure of coronavirus spikes in complex with carcinoembryonic antigen-related cell adhesion molecule cellular receptors, J Biol Chem, 277:19727-34.
18. Li et al., 2003, Angiotensin-converting enzyme 2 is a functional receptor for the SARS coronavirus, Nature, 426:4504.
19. Lin et al., 2001, Infectivity-neutralizing and hemagglutinin-inhibiting antibody responses to respiratory coronavirus infections of cattle in pathogenesis of shipping fever pneumonia, Clin Diagn Lab Immunol, 8:357-62.
20. Lu et al., 1998, Antigen engineering in DNA immunization. In: Methods in Molecular Medicine, Edited by Lowrie D B, Whalen R G, 29:355-74.
21. Lu et al., and T. Block, 1995, Evidence that N-linked glycosylation is necessary for hepatitis B virus secretion, Virology, 213:660-5.
22. Luo, Z., and S. R. Weiss, 1998, Roles in cell-to-cell fusion of two conserved hydrophobic regions in the murine coronavirus spike protein, Virology, 244:483-94.
23. Maley et al., 1989, Characterization of glycoproteins and their associated oligosaccharides through the use of endoglycosidases, Anal Biochem, 180:195-204.
24. Marra et al., 2003, The Genome sequence of the SARS-associated coronavirus, Science, 300:1399-404.
25. Mondal, S. P., and S. A. Naqi, 2001, Maternal antibody to infectious bronchitis virus: its role in protection against infection and development of active immunity to vaccine, Vet Immunol Immunopathol, 79:31-40.
26. Montefiori et al., 1998, Evidence that antibody-mediated neutralization of human immunodeficiency virus type 1 by sera from infected individuals is independent of coreceptor usage, J Virol, 72:1886-93.
27. Montefiori et al., 1988, Evaluation of antiviral drugs and neutralizing antibodies to human immunodeficiency virus by a rapid and sensitive microtiter infection assay, J Clin Microbiol, 26:231-5.
28. Moore et al., 2001, Genetic subtypes, humoral immunity, and human immunodeficiency virus type 1 vaccine development, J Virol, 75:5721-9.
29. Moore, J. P., and J. Sodroski, 1996, Antibody cross-competition analysis of the human immunodeficiency virus type 1 gp120 exterior envelope glycoprotein, J Virol, 70:1863-72.
30. Morrison, T. G., 2001, The three faces of paramyxovirus attachment proteins, Trends Microbiol, 9:103-5.
31. Oxford et al., 2003, Treatment of epidemic and pandemic influenza with neuraminidase and M2 proton channel inhibitors, Clin Microbiol Infect, 9:1-14.
32. Peiris et al., 2003, Coronavirus as a possible cause of severe acute respiratory syndrome, Lancet, 361:1319-25.
33. Qiu et al., and X. F. Yu, 2000, Enhancement of primary and secondary cellular immune responses against human immunodeficiency virus type 1 gag by using DNA expression vectors that target Gag antigen to the secretory pathway, J Virol, 74:5997-6005.
34. Rosenthal et al., 1998, Structure of the haemagglutinin-esterase-fusion glycoprotein of influenza C virus, Nature, 396:92-6.
35. Rota et al., 2003, Characterization of a novel coronavirus associated with severe acute respiratory syndrome, Science, 300:1394-9.
36. Saif, L. J., 1993, Coronavirus immunogens, Vet Microbiol, 37:285-97.
37. Sanchez et al., 1999, Targeted recombination demonstrates that the spike gene of transmissible gastroenteritis coronavirus is a determinant of its enteric tropism and virulence, J Virol, 73:7607-18.
38. Spiga et al., 2003, Molecular modelling of S1 and S2 subunits of SARS coronavirus spike glycoprotein, Biochem Biophys Res Commun, 310:78-83.
39. Sui et al., 2004, Potent neutralization of severe acute respiratory syndrome (SARS) coronavirus by a human mAb to S1 protein that blocks receptor association, Proc Natl Acad Sci U S A.
40. Taguchi, F., 2001, Mouse hepatitis virus (MHV) receptor and its interaction with MHV spike protein, Uirusu, 51:177-83.
41. Tarentino et al., Jr., 1985, Deglycosylation of asparagine-linked glycans by peptide:N-glycosidase F, Biochemistry, 24:4665-71.
42. Wang et al., 2004, A DNA vaccine producing LcrV antigen in oligomers is effective in protecting mice from lethal mucosal challenge of plague, Vaccine:in press.
43. Wang et al., 2004, Delivery of DNA to skin by particle bombardment, Methods Mol Biol, 245:185-96.
44. Wang et al., 2002, Construction and immunogenicity studies of recombinant fowl poxvirus containing the S1 gene of Massachusetts 41 strain of infectious bronchitis virus, Avian Dis, 46:831-8.
45. Weiss, C. D., 2003, HIV-1 gp41: mediator of fusion and target for inhibition, AIDS Rev, 5:214-21.
46. Weissenhorn et al., 1997, Atomic structure of the ectodomain from HIV-1 gp41, Nature, 387:426-30.
47. Wong et al., 2004, A 193-amino acid fragment of the SARS coronavirus S protein efficiently binds angiotensin-converting enzyme 2, J Biol Chem, 279:3197-201.
48. Yang et al., 2004, A DNA vaccine induces SARS coronavirus neutralization and protective immunity in mice, Nature, 428:561-4.
49. Ying et al., 2004, Proteomic analysis on structural proteins of Severe Acute Respiratory Syndrome coronavirus, Proteomics, 4:492-504.
50. Zelus et al., 2003, Conformational changes in the spike glycoprotein of murine coronavirus are induced at 37 degrees C. either by soluble murine CEACAM1 receptors or by pH 8, J Virol, 77:830-40.
51. Zolla-Pazner, S., 2004, Identifying epitopes of HIV-1 that induce protective antibodies, Nat Rev Immunol, 4:199-210.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. An isolated nucleic acid comprising:
a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, wherein the sequence has been codon-optimized for expression in a mammalian host.

2. The nucleic acid of claim 1 comprising:
a sequence encoding a SARS Co-V S polypeptide or fragment thereof, wherein the sequence comprises at least 95% identity with the sequence set forth in SEQ ID NO:1.

3. The nucleic acid of claim 1, wherein the sequence encodes a leader peptide that is not naturally associated with the SARS-CoV polypeptide.

4. The nucleic acid of claim 3, wherein the sequence encodes a tPA leader peptide.

5. The nucleic acid of claim 2, wherein the sequence comprises at least 95% identity with the sequence set forth in SEQ ID NO:3 or SEQ ID NO:5.

6. The nucleic acid of claim 2, wherein the sequence encodes an extracellular portion of the S polypeptide.

7. The nucleic acid of claim 2, wherein the sequence has less than 99% identity with a naturally circulating variant sequence encoding the SARS-CoV S polypeptide.

8. The nucleic acid of claim 2, wherein the sequence has less than 99% identity with SEQ ID NO:17.

9. The nucleic acid of claim 2, wherein the sequence differs from SEQ ID NO:17 by at least 20, 30, 40, 50, or 100 nucleotides.

10. The nucleic acid of claim 2, wherein the sequence comprises SEQ ID NO:1 or SEQ ID NO:3.

11. The nucleic acid of claim 1 comprising:
a sequence encoding a SARS-CoV M polypeptide, or fragment thereof, wherein the sequence comprises at least 95% identity with the sequence set forth in SEQ ID NO:11.

12. The nucleic acid of claim 11, wherein the sequence comprises at least 95% identity with the sequence set forth in SEQ ID NO:11.

13. The nucleic acid of claim 11, wherein the sequence has less than 99% identity with a naturally circulating variant sequence encoding the SARS-CoV M polypeptide.

14. The nucleic acid of claim 1 1, wherein the sequence does not have 100% identity with SEQ ID NO:19.

15. The nucleic acid of claim 11, wherein the sequence differs from SEQ ID NO:19 by at least 20, 30, 40, 50, or 100 nucleotides.

16. The nucleic acid of claim 11, wherein the sequence comprises SEQ ID NO:11.

17. The nucleic acid of claim 1 comprising:
a sequence encoding a SARS-CoV E polypeptide, or fragment thereof, wherein the sequence comprises at least 95% identity with the sequence set forth in SEQ ID NO:13.

18. The nucleic acid of claim 17, wherein the sequence encodes an extracellular portion of the E polypeptide.

19. The nucleic acid of claim 17, wherein the sequence has less than 99% identity with a naturally circulating variant sequence encoding the SARS-CoV E polypeptide.

20. The nucleic acid of claim 17, wherein the sequence has less than 99% identity with SEQ ID NO:21.

21. The nucleic acid of claim 17, wherein the sequence differs from SEQ ID NO:21 by at least 20, 30, or 40 nucleotides.

22. The nucleic acid of claim 17, wherein the sequence comprises SEQ ID NO:13.

23. The nucleic acid of claim 1 comprising:
a sequence encoding a SARS-CoV N polypeptide, or fragment thereof, wherein the sequence comprises at least 95% identity with the sequence set forth in SEQ ID NO:15.

24. The nucleic acid of claim 23, wherein the sequence has less than 99% identity with a naturally circulating variant sequence encoding the SARS-CoV N polypeptide.

25. The nucleic acid of claim 23, wherein the sequence has less than 99% identity with SEQ ID NO:23.

26. The nucleic acid of claim 23, wherein the sequence differs from SEQ ID NO:23 by at least 20, 30, 40, 50, or 100 nucleotides.

27. The nucleic acid of claim 23, wherein the sequence comprises SEQ ID NO:15.

28. The nucleic acid of claim 1, wherein the sequence is operably linked to a promoter.

29. A nucleic acid expression vector comprising:
a sequence encoding a SARS-CoV S polypeptide, M polypeptide, E polypeptide, N polypeptide, or fragment thereof, wherein the sequence is codon-optimized for expression in a host cell.

30-33. (canceled)

34. A composition comprising an isolated nucleic acid, wherein the isolated nucleic acid comprises
(a) a codon-optimized sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof;
(b) a start codon immediately upstream of the nucleotide sequence;
(c) a mammalian promoter operably linked to the codon-optimized sequence; and
(d) a mammalian polyadenylation signal operably linked to the nucleotide sequence, wherein the promoter directs transcription of mRNA encoding the SARS-CoV polypeptide.

35. The composition of claim 34, further comprising an adjuvant.

36-38. (canceled)

39. The composition of claim 34, further comprising particles to which the isolated nucleic acid is bound, wherein the particles are suitable for intradermal, intramuscular or mucosal administration.

40. An isolated cell comprising the nucleic acid of claim 1.

41. The cell of claim 40, wherein the cell is a eukaryotic cell.

42. The cell of claim 41, wherein the cell is a mammalian cell.

43. The cell of claim 42, wherein the cell is a human cell.

44. An isolated polypeptide encoded by the nucleic acid of claim 1.

45. The polypeptide of claim 44, wherein the polypeptide is produced in a mammalian cell.

46. The polypeptide of claim 45, wherein the polypeptide is produced in a human cell.

47. An isolated antibody or antigen binding fragment thereof that specifically binds to a polypeptide of claim 44.

48. The antibody of claim 47, wherein the antibody is a polyclonal antibody.

49. The antibody of claim 47, wherein the antibody is a monoclonal antibody.

50. A method for making a SARS-CoV polypeptide, the method comprising:
constructing a nucleic acid, wherein the nucleic acid comprises a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, and wherein the codons encoding the polypeptide are optimized for expression in a host cell,
expressing the nucleic acid in the host cell under conditions that allow the polypeptide to be produced, and
isolating the polypeptide.

51. The method of claim 50, wherein the host cell is a mammalian cell.

52. A method for inducing an immune response to SARS-CoV polypeptide in a subject, the method comprising:
administering to the subject a composition comprising an isolated nucleic acid, wherein the isolated nucleic acid comprises
(a) a sequence encoding a SARS-CoV S polypeptide or fragment thereof, a SARS-CoV M polypeptide or fragment thereof, a SARS-CoV E polypeptide or fragment thereof, or a SARS-CoV N polypeptide or fragment thereof, wherein the sequence has been codon-optimized for expression in a mammalian host;
(b) a start codon immediately upstream of the nucleotide sequence;
(c) mammalian promoter operably linked to the codon-optimized sequence; and
(d) a mammalian polyadenylation signal operably linked to the nucleotide sequence, wherein the promoter directs transcription of mRNA encoding the SARS-CoV polypeptide, wherein the composition is administered in an amount sufficient for the nucleic acid to express the SARS-CoV polypeptide at a level sufficient to induce an immune response against the polypeptide in the subject.

53-54. (canceled)


SEQUENCE LISTING (24 sequences)

SEQ ID NO: 1 - DNA, 3768 bp, Artificial Sequence
  Codon-optimized nucleic acid sequence
atgttcatcttcctgctgttcctcaccctcaccagcggcagcgatctggataggtgcaccaccttcgacgacgtgcaggcccccaactacacccagcacaccagcagcatgaggggcgtgtactaccccgacgagatattcagaagcgacaccctgtacctcacccaggacctgttcctgcccttctacagcaacgtgaccggcttccacaccatcaaccacaccttcggcaaccccgtgatccctttcaaggacggcatctacttcgccgccaccgagaagagcaatgtggtgcggggctgggtgttcggcagcaccatgaacaacaagagccagagcgtgatcatcatcaacaacagcaccaacgtggtgatccgggcctgcaatttcgagctgtgcgacaaccctttcttcgccgtgtccaaacctatgggcacccagacccacaccatgatcttcgacaacgccttcaactgcaccttcgagtacatcagcgacgccttcagcctggatgtgagcgagaagagcggcaacttcaagcacctgcgggagttcgtgttcaagaacaaggacggcttcctgtacgtgtacaagggctaccagcccatcgacgtggtgagagacctgcccagcggcttcaacaccctgaagcccatcttcaagctgcccctgggcatcaacatcaccaacttccgggccatcctcaccgcctttagccctgcccaggatatctggggcaccagcgccgctgcctacttcgtgggctacctgaagcctaccaccttcatgctgaagtacgacgagaacggcaccatcaccgatgccgtggactgcagccagaaccccctggccgagctgaagtgcagcgtgaagagcttcgagatcgacaagggcatctaccagaccagcaacttcagagtggtgcctagcggcgatgtggtgaggttccccaatatcaccaacctgtgccccttcggcgaggtgttcaacgccaccaagttccctagcgtgtacgcctgggagcggaagaagatcagcaactgcgtggccgattacagcgtgctgtacaactccaccttcttcagcaccttcaagtgctacggcgtgagcgccaccaagctgaacgacctgtgcttcagcaacgtgtacgccgacagcttcgtggtgaagggcgacgacgtgagacagatcgcccctggccagaccggcgtgatcgccgactacaactacaagctgcccgacgacttcatgggctgcgtgctggcctggaacaccagaaacatcgacgccacctccaccggcaactacaattacaagtaccgctacctgaggcacggcaagctgagacccttcgagcgggacatctccaacgtgcccttcagccccgacggcaagccctgcaccccccctgccctgaactgctactggcccctgaacgactacggcttctacaccaccaccggcatcggctatcagccctacagagtggtggtgctgagcttcgagctgctgaacgcccctgccaccgtgtgcggccccaagctgagcaccgacctcatcaagaaccagtgcgtgaacttcaacttcaacggcctcaccggtaccggcgtgctcacccctagcagcaagaggttccagcccttccagcagttcggcagggacgtgagcgatttcaccgacagcgtgagggaccccaagaccagcgagatcctggacatcagcccttgcagcttcggcggcgtgagcgtgatcacccccggcaccaacgccagcagcgaggtggccgtgctgtaccaggacgtgaactgcaccgacgtgagcaccgccatccacgccgaccagctcacccccgcctggagaatctacagcaccggcaacaacgtgttccagacccaggccggctgcctcatcggcgccgagcacgtggacaccagctacgagtgcgacatccccatcggagccggcatctgcgccagctaccacaccgtgagcctgctgagaagcaccagccagaagagcatcgtggcctacaccatgagcctgggcgccgacagcagcatcgcctacagcaacaacaccatcgccatccccaccaacttcagcatcagcatcaccaccgaggtgatgcccgtgagcatggccaagacaagcgtggactgcaacatgtacatctgcggcgacagcaccgagtgcgccaacctgctgctgcagtacggcagcttctgcacccagctgaacagagccctgagcggcattgccgccgagcaggacagaaacaccagggaggtgttcgcccaggtgaagcagatgtataagacccccaccctgaagtacttcggcggcttcaacttcagccagatcctgcccgatcctctgaagcccaccaagagatctttcatcgaggacctgctgttcaacaaggtgaccctggccgacgccggctttatgaagcagtacggcgagtgcctgggcgatatcaacgccagggacctcatctgcgcccagaagttcaatggcctcaccgtgctgccccccctgctcaccgacgacatgatcgccgcctacacagccgccctggtgagcggcaccgccaccgccggctggacctttggcgccggagccgccctgcagatccccttcgccatgcagatggcctaccggttcaatggcatcggcgtgacccagaacgtgctgtacgagaaccagaagcagatcgccaaccagttcaataaggccatcagccagatccaggagagcctcaccaccacaagcaccgccctgggcaagctgcaggacgtggtgaaccagaacgcccaggccctgaataccctggtgaagcagctgagcagcaacttcggcgccatcagcagcgtgctgaacgacatcctgagcaggctggataaggtggaggccgaggtgcagatcgacagactcatcaccggcagactgcagagcctgcagacctacgtgacccagcagctcatcagagccgccgagatcagagccagcgccaacctggccgccaccaagatgagcgagtgcgtgctgggccagagcaagagagtggacttctgcggcaagggctaccacctcatgagcttcccccaggccgctccccacggcgtggtgttcctgcacgtgacctacgtgcctagccaggagaggaatttcaccaccgcccctgccatctgccacgagggcaaggcctacttccccagagagggcgtgttcgtgttcaacggcaccagctggttcatcacccagcggaacttcttcagcccccagatcatcaccaccgacaacaccttcgtgagcggcaactgcgacgtggtgatcggcatcatcaacaacaccgtgtacgaccctctgcagcctgagctggacagcttcaaggaggagctggacaagtacttcaagaaccacaccagccccgacgtggacctgggcgacatcagcggcatcaatgccagcgtggtgaacatccagaaggagatcgaccggctgaacgaggtggccaagaacctgaacgagagcctcatcgacctgcaggagctgggaaagtacgagcagtacatcaagtggccctggtacgtgtggctgggcttcatcgccggcctcatcgccatcgtgatggtgaccatcctgctgtgctgcatgaccagctgctgctcctgcctgaagggcgcctgcagctgtggcagctgctgcaagttcgacgaggacgacagcgagcccgtgctgaagggcgtgaagctgcactacacctga

SEQ ID NO: 2 - protein, 1255 aa, Artificial Sequence
  Synthetically generated peptide
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - DNA, 1608 bp, Artificial Sequence
  Codon-optimized  nucleic acid sequence
atgttcatcttcctgctgttcctcaccctcaccagcggcagcgatctggataggtgcaccaccttcgacgacgtgcaggcccccaactacacccagcacaccagcagcatgaggggcgtgtactaccccgacgagatattcagaagcgacaccctgtacctcacccaggacctgttcctgcccttctacagcaacgtgaccggcttccacaccatcaaccacaccttcggcaaccccgtgatccctttcaaggacggcatctacttcgccgccaccgagaagagcaatgtggtgcggggctgggtgttcggcagcaccatgaacaacaagagccagagcgtgatcatcatcaacaacagcaccaacgtggtgatccgggcctgcaatttcgagctgtgcgacaaccctttcttcgccgtgtccaaacctatgggcacccagacccacaccatgatcttcgacaacgccttcaactgcaccttcgagtacatcagcgacgccttcagcctggatgtgagcgagaagagcggcaacttcaagcacctgcgggagttcgtgttcaagaacaaggacggcttcctgtacgtgtacaagggctaccagcccatcgacgtggtgagagacctgcccagcggcttcaacaccctgaagcccatcttcaagctgcccctgggcatcaacatcaccaacttccgggccatcctcaccgcctttagccctgcccaggatatctggggcaccagcgccgctgcctacttcgtgggctacctgaagcctaccaccttcatgctgaagtacgacgagaacggcaccatcaccgatgccgtggactgcagccagaaccccctggccgagctgaagtgcagcgtgaagagcttcgagatcgacaagggcatctaccagaccagcaacttcagagtggtgcctagcggcgatgtggtgaggttccccaatatcaccaacctgtgccccttcggcgaggtgttcaacgccaccaagttccctagcgtgtacgcctgggagcggaagaagatcagcaactgcgtggccgattacagcgtgctgtacaactccaccttcttcagcaccttcaagtgctacggcgtgagcgccaccaagctgaacgacctgtgcttcagcaacgtgtacgccgacagcttcgtggtgaagggcgacgacgtgagacagatcgcccctggccagaccggcgtgatcgccgactacaactacaagctgcccgacgacttcatgggctgcgtgctggcctggaacaccagaaacatcgacgccacctccaccggcaactacaattacaagtaccgctacctgaggcacggcaagctgagacccttcgagcgggacatctccaacgtgcccttcagccccgacggcaagccctgcaccccccctgccctgaactgctactggcccctgaacgactacggcttctacaccaccaccggcatcggctatcagccctacagagtggtggtgctgagcttcgagctgctgaacgcccctgccaccgtgtgcggccccaagctgagcaccgacctcatcaagaaccagtgcgtgaacttcaacttcaacggcctcaccggtacctga

SEQ ID NO: 4 - protein, 535 aa, Artificial Sequence
  Synthetically generated peptide
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGT

SEQ ID NO: 5 - DNA, 1644 bp, Artificial Sequence
  Codon-optimized nucleic acid sequence
atggatgcaatgaagagagggctctgctgtgtgctgctgctgtgtggagcagtcttcgtttcggctagcagcggcagcgatctggataggtgcaccaccttcgacgacgtgcaggcccccaactacacccagcacaccagcagcatgaggggcgtgtactaccccgacgagatattcagaagcgacaccctgtacctcacccaggacctgttcctgcccttctacagcaacgtgaccggcttccacaccatcaaccacaccttcggcaaccccgtgatccctttcaaggacggcatctacttcgccgccaccgagaagagcaatgtggtgcggggctgggtgttcggcagcaccatgaacaacaagagccagagcgtgatcatcatcaacaacagcaccaacgtggtgatccgggcctgcaatttcgagctgtgcgacaaccctttcttcgccgtgtccaaacctatgggcacccagacccacaccatgatcttcgacaacgccttcaactgcaccttcgagtacatcagcgacgccttcagcctggatgtgagcgagaagagcggcaacttcaagcacctgcgggagttcgtgttcaagaacaaggacggcttcctgtacgtgtacaagggctaccagcccatcgacgtggtgagagacctgcccagcggcttcaacaccctgaagcccatcttcaagctgcccctgggcatcaacatcaccaacttccgggccatcctcaccgcctttagccctgcccaggatatctggggcaccagcgccgctgcctacttcgtgggctacctgaagcctaccaccttcatgctgaagtacgacgagaacggcaccatcaccgatgccgtggactgcagccagaaccccctggccgagctgaagtgcagcgtgaagagcttcgagatcgacaagggcatctaccagaccagcaacttcagagtggtgcctagcggcgatgtggtgaggttccccaatatcaccaacctgtgccccttcggcgaggtgttcaacgccaccaagttccctagcgtgtacgcctgggagcggaagaagatcagcaactgcgtggccgattacagcgtgctgtacaactccaccttcttcagcaccttcaagtgctacggcgtgagcgccaccaagctgaacgacctgtgcttcagcaacgtgtacgccgacagcttcgtggtgaagggcgacgacgtgagacagatcgcccctggccagaccggcgtgatcgccgactacaactacaagctgcccgacgacttcatgggctgcgtgctggcctggaacaccagaaacatcgacgccacctccaccggcaactacaattacaagtaccgctacctgaggcacggcaagctgagacccttcgagcgggacatctccaacgtgcccttcagccccgacggcaagccctgcaccccccctgccctgaactgctactggcccctgaacgactacggcttctacaccaccaccggcatcggctatcagccctacagagtggtggtgctgagcttcgagctgctgaacgcccctgccaccgtgtgcggccccaagctgagcaccgacctcatcaagaaccagtgcgtgaacttcaacttcaacggcctcaccggtacctga

SEQ ID NO: 6 - protein, 547 aa, Artificial Sequence
  Synthetically generated peptide
MDAMKRGLCCVLLLCGAVFVSASSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGT

SEQ ID NO: 7 - DNA, 879 bp, Artificial Sequence
  Codon-optimized  nucleic acid sequence
atggatgcaatgaagagagggctctgctgtgtgctgctgctgtgtggagcagtcttcgtttcggctagcggtaccggcgtgctcacccctagcagcaagaggttccagcccttccagcagttcggcagggacgtgagcgatttcaccgacagcgtgagggaccccaagaccagcgagatcctggacatcagcccttgcagcttcggcggcgtgagcgtgatcacccccggcaccaacgccagcagcgaggtggccgtgctgtaccaggacgtgaactgcaccgacgtgagcaccgccatccacgccgaccagctcacccccgcctggagaatctacagcaccggcaacaacgtgttccagacccaggccggctgcctcatcggcgccgagcacgtggacaccagctacgagtgcgacatccccatcggagccggcatctgcgccagctaccacaccgtgagcctgctgagaagcaccagccagaagagcatcgtggcctacaccatgagcctgggcgccgacagcagcatcgcctacagcaacaacaccatcgccatccccaccaacttcagcatcagcatcaccaccgaggtgatgcccgtgagcatggccaagacaagcgtggactgcaacatgtacatctgcggcgacagcaccgagtgcgccaacctgctgctgcagtacggcagcttctgcacccagctgaacagagccctgagcggcattgccgccgagcaggacagaaacaccagggaggtgttcgcccaggtgaagcagatgtataagacccccaccctgaagtacttcggcggcttcaacttcagccagatcctgcccgatcctctgaagcccaccaagagatcttgaggatccactcta

SEQ ID NO: 8 - protein, 288 aa, Artificial Sequence
  Synthetically generated peptide
MDAMKRGLCCVLLLCGAVFVSASGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRS

SEQ ID NO: 9 - DNA, 1461 bp, Artificial Sequence
  Codon-optimized  nucleic acid sequence
atggatgcaatgaagagagggctctgctgtgtgctgctgctgtgtggagcagtcttcgtttcggctagcagatctttcatcgaggacctgctgttcaacaaggtgaccctggccgacgccggctttatgaagcagtacggcgagtgcctgggcgatatcaacgccagggacctcatctgcgcccagaagttcaatggcctcaccgtgctgccccccctgctcaccgacgacatgatcgccgcctacacagccgccctggtgagcggcaccgccaccgccggctggacctttggcgccggagccgccctgcagatccccttcgccatgcagatggcctaccggttcaatggcatcggcgtgacccagaacgtgctgtacgagaaccagaagcagatcgccaaccagttcaataaggccatcagccagatccaggagagcctcaccaccacaagcaccgccctgggcaagctgcaggacgtggtgaaccagaacgcccaggccctgaataccctggtgaagcagctgagcagcaacttcggcgccatcagcagcgtgctgaacgacatcctgagcaggctggataaggtggaggccgaggtgcagatcgacagactcatcaccggcagactgcagagcctgcagacctacgtgacccagcagctcatcagagccgccgagatcagagccagcgccaacctggccgccaccaagatgagcgagtgcgtgctgggccagagcaagagagtggacttctgcggcaagggctaccacctcatgagcttcccccaggccgctccccacggcgtggtgttcctgcacgtgacctacgtgcctagccaggagaggaatttcaccaccgcccctgccatctgccacgagggcaaggcctacttccccagagagggcgtgttcgtgttcaacggcaccagctggttcatcacccagcggaacttcttcagcccccagatcatcaccaccgacaacaccttcgtgagcggcaactgcgacgtggtgatcggcatcatcaacaacaccgtgtacgaccctctgcagcctgagctggacagcttcaaggaggagctggacaagtacttcaagaaccacaccagccccgacgtggacctgggcgacatcagcggcatcaatgccagcgtggtgaacatccagaaggagatcgaccggctgaacgaggtggccaagaacctgaacgagagcctcatcgacctgcaggagctgggaaagtacgagcagtacatcaagtggccctggtacgtgtggctgggcttcatcgccggcctcatcgccatcgtgatggtgaccatcctgctgtgctgcatgaccagctgctgctcctgcctgaagggcgcctgcagctgtggcagctgctgcaagttcgacgaggacgacagcgagcccgtgctgaagggcgtgaagctgcactacacctgaggatccactcta

SEQ ID NO: 10 - protein, 482 aa, Artificial Sequence
  Synthetically generated peptide
MDAMKRGLCCVLLLCGAVFVSASRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - DNA, 666 bp, Artificial Sequence
  Codon-optimized  nucleic acid sequence
atggccgacaacggcaccatcaccgtggaggagctgaagcagctgctggagcagtggaacctggtgatcggcttcctgttcctggcctggatcatgctgctgcagttcgcctacagcaaccgcaacaggttcctgtacatcatcaagctggtgttcctgtggctgctgtggcccgtgaccctggcctgcttcgtgctggccgccgtgtaccgcatcaactgggtgaccggcggcattgccatcgccatggcctgcatcgtgggcctgatgtggctgagctacttcgtggcctccttccgcctgttcgcccgcacccgcagcatgtggagcttcaaccccgagaccaacatccttctgaacgtgcccctgcgcggcaccatcgtgacccgccccctgatggagagcgagctggtgatcggtgccgtgatcattcgcggccacctgcgcatggccggccaccccctgggccgctgcgacatcaaggacctgcccaaggagatcaccgtggctaccagccgcacgctgagctactacaagctgggagcctcgcagcgcgtgggcaccgatagcggcttcgccgcctacaaccgctaccgcatcggcaactacaagctgaacaccgaccacgccggcagcaacgacaacatcgccctgctggtgcagtaa

SEQ ID NO: 12 - protein, 222 aa, Artificial Sequence
  Synthetically generated peptide
  VARIANT 222: Xaa = any amino acid
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQX

SEQ ID NO: 13 - DNA, 231 bp, Artificial Sequence
  Codon-optimized nucleic acid sequence
atgtacagcttcgtgagcgaggagaccggcaccctgatcgtgaacagcgtgctgctgttcctggctttcgtggtgttcctgctggtgaccctggccatcctgaccgccctgcgcctgtgcgcctactgctgcaacatcgtgaacgtgagcctggtgaaacccaccgtgtacgtgtactcgcgcgtgaaaaacctgaacagcagcgagggcgtgcccgacctgctggtgtaa

SEQ ID NO: 14 - protein, 76 aa, Artificial Sequence
  Synthetically generated peptide
MYSFVSEETGTLIVNSVLLFLAFVVFLLVTLAILTALRLCAYCCNIVNVSLVKPTVYVYSRVKNLNSSEGVPDLLV

SEQ ID NO: 15 - DNA, 1272 bp, Artificial Sequence
  Codon-optimized nucleic acid sequence
atgagcgacaacggaccccagagcaaccagcgcagcgcccctcgcatcaccttcggcggacccaccgacagcaccgacaacaaccagaacggcggacgcaatggcgcaaggcccaagcagcgccgaccccaaggcttacccaacaacaccgccagctggttcacagccctgacccagcacggcaaggaggagctgcgcttccctcgcggccagggcgtgcccatcaacaccaacagtggcccagacgaccagatcggctactaccgcagagccacccgacgcgttcgcggtggcgacggcaagatgaaggagctgagccccagatggtacttctactacctaggcactggcccagaagccagccttccctacggcgctaacaaggagggcatcgtatgggttgccaccgagggcgccctgaacacacccaaagaccacattggcacccgcaatcccaacaacaacgctgccaccgtgctgcagctgcctcaaggcacaaccctgcccaagggcttctacgccgagggcagcagaggcggcagccaggccagcagccgcagcagcagccgcagccgcggcaacagccgcaacagcactcctggcagcagtcgcggcaactctcccgcacgcatggccagcggcggtggcgagactgccctggccctgttgctgctggaccgcctgaaccagctggagagcaaggtgagcggcaaaggccaacagcagcaaggccagaccgtgaccaagaagagcgctgccgaggcaagcaagaagccccgccagaagcgcactgccaccaagcagtacaacgtgacccaagccttcggcagacgcggacccgagcagacccagggcaacttcggcgaccaggacctgatccgccagggcaccgactacaagcactggccgcagatcgcacagttcgctcccagtgccagcgccttcttcggcatgagccgcattggcatggaggtgacacccagcggcacctggctgacctaccacggagccatcaagctggacgacaaggaccctcagttcaaggacaacgtgatcctgctgaacaagcacatcgacgcctacaagaccttcccacccaccgagcccaagaaggacaagaagaagaagaccgacgaggcccagcctctgccccagcgccagaagaagcagcccaccgtgaccctgcttcctgccgctgacatggatgacttcagccgccagctgcagaacagcatgagcggagcctctgccgacagcacccaggcataatga

SEQ ID NO: 16 - protein, 422 aa, Artificial Sequence
  Synthetically generated peptide
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 17 - DNA, 3768 bp, SARS coronavirus Urbani
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 18 - protein, 1255 aa, SARS coronavirus Urbani; identical to SEQ ID NO: 2

SEQ ID NO: 19 - DNA, 666 bp, SARS coronavirus Urbani
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaa

SEQ ID NO: 20 - protein, 221 aa, SARS coronavirus Urbani
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQ

SEQ ID NO: 21 - DNA, 231 bp, SARS coronavirus Urbani
atgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaa

SEQ ID NO: 22 - protein, 76 aa, SARS coronavirus Urbani; identical to SEQ ID NO: 14

SEQ ID NO: 23 - DNA, 1259 bp, SARS coronavirus Urbani
atggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 24 - protein, 422 aa, SARS coronavirus Urbani; identical to SEQ ID NO: 16